(19) United States
(12) Patent Application Publication
(10) Pub. No.: US 2026/0327175 A1
(43) Pub. Date: Oct. 22, 2026

(54) METHOD FOR PRODUCING (METH)ACRYLIC ACID COMPOUND

(71) Applicant: OSAKA ORGANIC CHEMICAL INDUSTRY LTD., Osaka (JP)

(72) Inventor: Ryoichi AKAISHI, Kashiwara-shi, Osaka (JP)

(21) Appl. No.: 19/478,695

(22) PCT Filed: Apr. 26, 2024
(86) PCT No.: PCT/JP2024/016469

§ 371 Date: Oct. 24, 2025

(30) Foreign Application Priority Data
Apr. 28, 2023 (JP) 2023-074625

(51) Int. Cl. C07C 51/09

(52) U.S. Cl. CPC C07C 51/09

(57) ABSTRACT

A method for producing a (meth)acrylic acid compound using a hydroxycarboxylic acid and/or a polymer thereof as a raw material, comprising at least a reaction step of heating a mixture containing a hydroxycarboxylic acid and/or a polymer thereof, a basic catalyst, and a carboxylic acid anhydride to react with each other and produce a (meth)acrylic acid compound.

### CROSS REFERENCE TO RELATED APPLICATIONS

This application is a national stage application under 35 U.S.C. § 371 and claims the benefit of PCT application no. PCT/JP2024/016469 having an international filing date of 26 Apr. 2024, which designated the United States, which PCT application claimed the benefit of Japanese patent application no. 2023-074625 filed 28 Apr. 2023. The disclosures of both before-mentioned applications are incorporated herein by reference in their entireties.

### TECHNICAL FIELD

### Technical Field

The present invention relates to a method for producing a (meth)acrylic acid compound.

### Background Art

The (meth)acrylic acid compound is a compound widely used as a raw material for synthesizing a poly(meth)acrylic acid resin. As a method for producing a (meth)acrylic acid compound, a method for producing a (meth)acrylic acid compound using a polymer of 3-hydroxycarboxylic acid as a raw material has been studied, for example, a method for producing (meth)acrylic acid by heating and/or contacting with a catalyst a composition containing a specific polymer of 3-hydroxycarboxylic acid (Patent Document 1), a method for producing (meth)acrylic acid by heating a raw material composition containing a predetermined amount of polymer of 3-hydroxycarboxylic acid to generate a gaseous decomposition product of the polymer of 3-hydroxycarboxylic acid and contacting the decomposition product with a dehydration catalyst (Patent Document 2), a method for producing (meth)acrylic acid by heating a raw material composition containing a predetermined amount of a polymer of 3-hydroxycarboxylic acid of trimer to eicosamer (Patent Document 3), and the like are known.

### PRIOR ART DOCUMENT

### Patent Documents

Patent Document 1: WO 2014/002886 A
Patent Document 2: JP-A-2014-156449
Patent Document 3: JP-A-2014-156450

### SUMMARY OF THE INVENTION

### Problems to be Solved by the Invention

All of the conventionally known methods for producing (meth)acrylic acid from a composition containing a polymer of 3-hydroxycarboxylic acid are those for producing (meth)acrylic acid by decomposing and dehydrating a polymer of 3-hydroxycarboxylic acid, and severe reaction conditions such as heating at a high temperature or using a strong dehydration catalyst are required.

An object of the present invention is to provide a novel production method capable of producing a (meth)acrylic acid compound from a hydroxycarboxylic acid and/or a polymer thereof.

### Solutions to the Problems

The present inventors conducted extensive research into a method for producing (meth)acrylic acid from a composition containing a hydroxycarboxylic acid and/or a polymer thereof, and as a result discovered that a (meth)acrylic acid compound can be produced by heating and reacting a mixture containing a hydroxycarboxylic acid and/or a polymer thereof, a basic catalyst, and a carboxylic acid anhydride, thus completing the present invention.

That is, the present invention relates to the following [1] to [16].
[1]A method for producing a (meth)acrylic acid compound using a hydroxycarboxylic acid and/or a polymer thereof as a raw material, comprising at least a reaction step of heating a mixture containing a hydroxycarboxylic acid and/or a polymer thereof and a basic catalyst to react with each other to produce a (meth)acrylic acid compound, wherein (1) the mixture further contains a carboxylic acid anhydride, and/or (2) the weight-average molecular weight of the polymer of the hydroxycarboxylic acid contained in the raw material is 10,000 or more.
[2] The method according to [1], a method for producing a (meth)acrylic acid compound using a 3-hydroxycarboxylic acid and/or a polymer thereof as a raw material, comprising at least a reaction step of heating a mixture containing a 3-hydroxycarboxylic acid and/or a polymer thereof, a basic catalyst, and a carboxylic acid anhydride to react with each other to produce a (meth)acrylic acid compound.
[3] The method according to [1], wherein the hydroxycarboxylic acid is at least one selected from the group consisting of 2-hydroxycarboxylic acid and 3-hydroxycarboxylic acid.
[4] The method according to any one of [1] to [3], wherein the carboxylic acid anhydride is (meth)acrylic acid anhydride.
[5] The method according to [4], wherein the (meth)acrylic anhydride is represented by the formula (I):

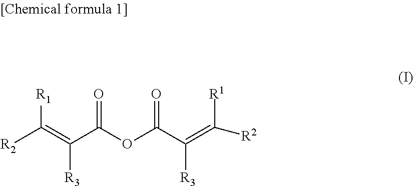

wherein
R¹ to R³ represent, each independently, a hydrogen atom or an alkyl group having 1 to 3 carbon atoms.
[6] The method according to any one of [1] to [5], wherein the basic catalyst is selected from the group consisting of an alkali metal carboxylate, a quaternary ammonium carboxylate, and an ammonium betaine.
[7] The method according to any one of [1] to [6], wherein the basic catalyst is at least one alkali metal carboxylate selected from the group consisting of an alkali metal salt of an α,β-ethylenically unsaturated monocarboxylic acid and an alkali metal salt of an aliphatic monocarboxylic acid.
[8] The method according to any one of [1] to [7], wherein the basic catalyst is at least one alkali metal carboxylate selected from the group consisting of alkali metal (meth)acrylate and alkali metal acetate.
[9] The method according to any one of [1] to [8], wherein the amount of the basic catalyst is 0.1 to 20 parts by mass per 100 parts by mass of the mixture including the hydroxycarboxylic acid and its polymer.
[10] The method according to any one of [1] to [9], wherein the amount of the carboxylic acid anhydride is 1 to 50 parts by mass per 100 parts by mass of the total of the hydroxycarboxylic acid and the polymer thereof in the mixture.
[11] The method according to any one of [1] to [10], wherein the heating temperature in the reaction step is 80 to 220 °C.
[12] The process according to any one of [1] to [11], wherein the hydroxycarboxylic acid is 3-hydroxypropionic acid, and the carboxylic acid anhydride is a (meth)acrylic acid anhydride represented by the formula (I) in which R¹ to R³ is a hydrogen atom, or R¹ and R² are a hydrogen atom, and R³ represents a methyl group.
[13] The method according to any one of [1] to [12], wherein the mixture further comprises a polymerization inhibitor.
[14] The method according to any one of [1] to [13], wherein the content of the solvent in the mixture is 0 to 50% by mass based on the total amount of the mixture.
[15] The method according to any one of [1] to [14], wherein the weight-average molecular weight of the polymer of the hydroxycarboxylic acid contained in the raw material is 10,000 or more, and the content of water in the mixture is 0 to 15% by mass based on the total amount of the mixture.
[16] The method according to any one of [1] to [15], further comprising a step of distilling off the (meth)acrylic acid compound produced in the reaction step.

### Effects of the Invention

According to the present invention, it is possible to provide a novel production method capable of producing a (meth)acrylic acid compound from a hydroxycarboxylic acid and/or a polymer thereof.

### BRIEF DESCRIPTION OF THE DRAWINGS

FIG. 1 is a diagram showing a HPLC chart of the raw material (A) containing 3-hydroxypropionic acid and its polymer obtained in Preparation Example 1.

FIG. 2 is a diagram showing an HPLC chart of reaction product (1) obtained in Example 1.

FIG. 3 is a diagram showing an HPLC chart of the distillate obtained in Example 3.

FIG. 4 is a diagram showing a ¹H-NMR chart of the distillate obtained in Example 3.

FIG. 5 is a diagram showing a HPLC chart of reaction product (3) obtained in Example 3.

FIG. 6 is a diagram showing a ¹H-NMR chart of the reaction product (3) obtained in Example 3.

### DETAILED DESCRIPTION

Hereinafter, embodiments of the present invention will be described in detail. The scope of the present invention is not limited to the embodiments described herein, and various modifications can be made without departing from the spirit of the present invention.

The method for producing a (meth)acrylic acid compound using a hydroxycarboxylic acid and/or a polymer thereof as a raw material, comprising at least a reaction step of heating a mixture containing a hydroxycarboxylic acid and/or a polymer thereof and a basic catalyst to react with each other to produce a (meth)acrylic acid compound, wherein (1) the mixture further contains a carboxylic acid anhydride, and/or (2) the weight average molecular weight of the polymer of the hydroxycarboxylic acid contained in the raw material is 10,000 or more. In the production method of the present invention, by heating the mixture, the raw material hydroxycarboxylic acid and/or its polymer and the basic catalyst react with the carboxylic acid anhydride, as the case may be, to produce a (meth)acrylic acid compound. The production method of the present invention may satisfy the above (1), may satisfy (2), or may satisfy both (1) and (2). Examples of the hydroxycarboxylic acid include 2-hydroxycarboxylic acid and 3-hydroxycarboxylic acid. In the production method of the present invention, when (1) is satisfied, 2-hydroxycarboxylic acids and polymers thereof, 3-hydroxycarboxylic acids and polymers thereof, polymers of 2-hydroxycarboxylic acids and 3-hydroxycarboxylic acids, and mixtures thereof can be preferably used. When (2) is satisfied, polymers of 2-hydroxycarboxylic acids having a weight-average molecular weight of 10,000 or more, polymers of 3-hydroxycarboxylic acids having a weight-average molecular weight of 10,000 or more, polymers of 2-hydroxycarboxylic acids and 3-hydroxycarboxylic acids having a weight-average molecular weight of 10,000 or more, and mixtures thereof can be preferably used.

According to the present inventors, in the method for producing a (meth)acrylic acid compound using a hydroxycarboxylic acid and/or a polymer thereof as a raw material, it was found that the reaction for giving a (meth)acrylic acid compound from a hydroxycarboxylic acid and/or a polymer thereof used as a raw material proceeds efficiently when (1) a carboxylic acid anhydride is further included and/or (2) the weight-average molecular weight of the polymer of the hydroxycarboxylic acid contained in the raw material is 10,000 or more.

In one aspect, the production method of the present invention is a production method for a (meth)acrylic acid compound using a 3-hydroxycarboxylic acid and/or its polymer as a raw material, comprising at least a reaction step of heating a mixture containing a 3-hydroxycarboxylic acid and/or its polymer, a basic catalyst, and a carboxylic acid anhydride to react with each other to produce a (meth)acrylic acid compound.

In the production method of the present invention, a (meth)acrylic acid compound can be produced by reacting a hydroxycarboxylic acid as a raw material and/or a polymer thereof, a basic catalyst, and a carboxylic acid anhydride by heating the mixture. First, an embodiment (1) in which a mixture containing a hydroxycarboxylic acid and/or a polymer thereof and a basic catalyst further contains a carboxylic acid anhydride will be described.

(Hydroxycarboxylic Acid and/or Polymer Thereof)

The hydroxycarboxylic acid and/or the polymer thereof is a component that serves as a raw material for providing a (meth)acrylic acid compound in the production method of the present invention. In the production method of the present invention, at least one of a hydroxycarboxylic acid and/or a polymer thereof may be used, only a hydroxycarboxylic acid may be used, only a polymer of a hydroxycarboxylic acid may be used, or a mixture of a hydroxycarboxylic acid and a polymer of a hydroxycarboxylic acid may be used. The hydroxycarboxylic acid may be one hydroxycarboxylic acid or a mixture of two or more hydroxycarboxylic acids. The polymer of the hydroxycarboxylic acid may also be a polymer of one kind or two or more kinds of hydroxycarboxylic acids, and the degree of polymerization may be one kind or two or more kinds.

The 3-hydroxycarboxylic acid is a compound having at least a carboxylic acid group and a hydroxyl group at the β-position of the carbonyl carbon of the carboxylic acid group, and examples of the 3-hydroxycarboxylic acid include 3-hydroxypropionic acid, 3-hydroxybutanoic acid, 3-hydroxypentanoic acid, 3-hydroxyhexanoic acid, and a compound in which a hydrogen atom bonded to a carbon atom of the 3-hydroxycarboxylic acid is substituted with a substituent such as an alkyl group. The polymer of 3-hydroxycarboxylic acid is, for example, a polymer in which two or more 3-hydroxycarboxylic acids are bonded to each other via an intermolecular ester bond.

The 2-hydroxycarboxylic acid is a compound having at least a carboxylic acid group and a hydroxyl group at the α-position of the carbonyl carbon of the carboxylic acid group, and examples of the 2-hydroxycarboxylic acid include 2-hydroxypropionic acid, 2-hydroxybutanoic acid, 2-hydroxyisobutanoic acid, 2-hydroxypentanoic acid, 2-hydroxyhexanoic acid, and a compound in which a hydrogen atom bonded to a carbon atom of the 2-hydroxycarboxylic acid is substituted with a substituent such as an alkyl group. The polymer of 2-hydroxycarboxylic acid is, for example, a polymer in which two or more 2-hydroxycarboxylic acids are bonded to each other via intermolecular ester bonds.

The polymer of the hydroxycarboxylic acid may be a polymer obtained by polymerizing two or more hydroxycarboxylic acids, and is, for example, a multimer obtained by ester-bonded two or more hydroxycarboxylic acids.

The degree of polymerization and distribution of the multimer are not particularly limited. The polymer of the hydroxycarboxylic acid may be composed of one type of hydroxycarboxylic acid, may be composed of two or more types of hydroxycarboxylic acids, or may contain a structural unit other than the hydroxycarboxylic acid. From the viewpoint of efficient production of (meth)acrylic acid compounds, it is preferable that the polymer of hydroxycarboxylic acid contains only hydroxycarboxylic acid as a structural unit.

In one embodiment, when the mixture containing the hydroxycarboxylic acid and/or the polymer thereof and the basic catalyst further contains a carboxylic acid anhydride, the degree of polymerization of the polymerized product of the hydroxycarboxylic acid that may be contained in the mixture is not particularly limited, and a mixture having various degrees of polymerization may be used as a raw material. From the viewpoint of availability and manufacturability, the degree of polymerization of the hydroxycarboxylic acid may be, for example, dimer to 30,000 mer The content of the dimer to 30,000 mer of hydroxycarboxylic acid relative to a total of 100 parts by mass of hydroxycarboxylic acid and/or polymer thereof is preferably 70% by mass or more, more preferably 80% by mass or more, and even more preferably 90% by mass or more. From the same viewpoint, the content of the dimer to 20,000 mer, more preferably dimer to 15,000 mer, and even more preferably dimer to 10,000 mer of 3-hydroxycarboxylic acid is preferably 70% by mass or more, more preferably 80% by mass or more, and even more preferably 90% by mass or more relative to a total of 100 parts by mass of the hydroxycarboxylic acid and polymer thereof. The content of each polymer relative to 100 parts by mass of the total of the hydroxycarboxylic acid and polymer thereof can be calculated, for example, by HPLC, ¹H-NMR, or the like.

In a preferred embodiment, from the viewpoint of the production efficiency of the (meth)acrylic acid compound, the content of the dimer to 20 mer of the hydroxycarboxylic acid is preferably 70% by mass or more, more preferably 80% by mass or more, and still more preferably 90% by mass or more, based on 100 parts by mass of the total of the hydroxycarboxylic acid and the polymer thereof.

From the same viewpoint, The content of preferably dimer to 15 mer, more preferably dimer to 10 mer of hydroxycarboxylic acid is preferably 70% by mass or more, more preferably 80% by mass or more, and even more preferably 90% by mass or more, based on 100 parts by mass of the total of hydroxycarboxylic acid and polymer thereof. The content of each multimer with respect to 100 parts by mass of the sum of the hydroxycarboxylic acid and the polymer thereof can be calculated from, for example, HPLC and ¹H-NMR.

The 3-hydroxycarboxylic acid and its polymer may be, for example, a compound represented by formula (III)-1:

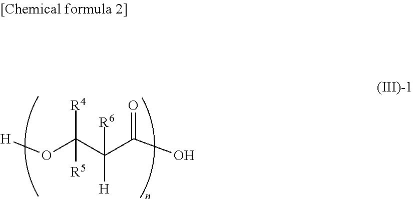

wherein
R⁴ to R⁶ each independently represents a hydrogen atom or an alkyl group having 1 to 3 carbon atoms, and n represents an integer of 1 or more.

When n in formula (III)-1 is 1, the 3-hydroxycarboxylic acid represented by formula (II)-1:

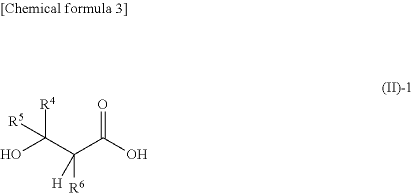

wherein
R⁴ to R⁶ each independently represent a hydrogen atom or an alkyl group having 1 to 3 carbon atoms. It is expressed by.
The compound in formula (III)-1 wherein n is 2 or more is a polymer of 3-hydroxycarboxylic acid represented by formula (II)-1.

In formula (II)-1 and formula (III)-1, R⁴ to R⁶ each independently represent a hydrogen atom or an alkyl group having 1 to 3 carbon atoms. Examples of alkyl group having 1 to 3 carbon atoms include a methyl group, an ethyl group, a n-propyl group, and an isopropyl group. R⁴ to R⁶ each independently represent preferably a hydrogen atom or an alkyl group having 1 to 2 carbon atoms, more preferably a hydrogen atom or a methyl group, and still more preferably a hydrogen atom. In a preferred embodiment, R⁵, R⁶ represents a hydrogen atom or an alkyl group having 1 to 3 carbon atoms. and R⁴ represents a hydrogen atom. In this case, R⁵, R⁶ preferably represents a hydrogen atom or an alkyl group having 1 to 2 carbon atoms, more preferably a hydrogen atom or a methyl group, and still more preferably a hydrogen atom. The 3-hydroxycarboxylic acid of the formula (II)-1 in which R⁴ to R⁶ represents a hydrogen atom is also referred to as 3-hydroxypropionic acid.

The n in formula (III)-1 is not particularly limited as long as it is 1 or more, and it is considered that 3-hydroxycarboxylic acid and polymer thereof usually contain multiple types of compounds or polymers with different n. The upper limit of n is not particularly limited, and may be, for example, 30,000 or less, preferably 20,000 or less, more preferably 10,000 or less, 9000 or less, 8000 or less, 7000 or less, 5000 or less, 3,000 or less, 1,000 or less, 500 or less, or the like. The upper limit of n is not particularly limited, but from the viewpoint of easily enhancing the effect of further including carboxylic acid anhydride, it may be, for example, 100 or less, 80 or less, 60 or less, 50 or less, 40 or less, 30 or less, etc. In one preferred embodiment of the present invention, from the viewpoint of production efficiency of the (meth)acrylic acid compound, in the 3-hydroxycarboxylic acid represented by formula (III)-1 and polymer thereof, the ratio of the polymer of the 3-hydroxycarboxylic acid represented by formula (III)-1, preferably n=2 to 30,000, more preferably n=2 to 20,000, even more preferably n=2 to 15,000, and even more preferably 2 to 10,000, to 100 parts by mass of the total of the 3-hydroxycarboxylic acid and polymer thereof is preferably 70 mol % or more, more preferably 80 mol % or more, and even more preferably 90 mol % or more. In addition, from the viewpoint of easily enhancing the effect of further containing a carboxylic acid anhydride, the ratio of the polymer of the 3-hydroxycarboxylic acid represented by formula (III)-1, preferably n=2 to 20, more preferably n=2 to 15, and even more preferably n=2 to 10, to 100 parts by mass of the total of the 3-hydroxycarboxylic acid and its polymer is preferably 70 mol % or more, more preferably 80 mol % or more, and even more preferably 90 mol % or more.

The 2-hydroxycarboxylic acid and its polymer may be, for example, a compound represented by formula (III)-2:

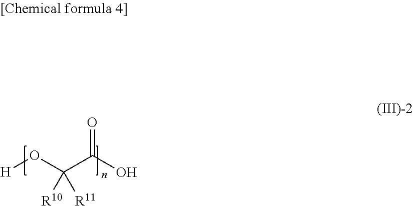

Wherein
R¹⁰ and R¹¹ each independently represent a hydrogen atom or an alkyl group having 1 to 3 carbon atoms, and n represents an integer of 1 or more.

When n in formula (III)-2 is 1, the 2-hydroxycarboxylic acid is represented by formula (II)-2:

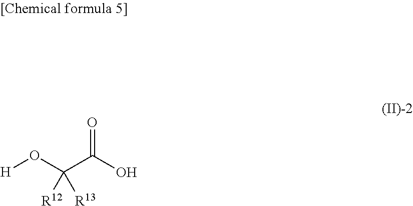

Wherein
R¹² and R¹³ each independently represent a hydrogen atom or an alkyl group having 1 to 3 carbon atoms. The compound in formula (III)-2 in wherein n is 2 or more is a polymer of 2-hydroxycarboxylic acid represented by formula (II)-2.

In formula (II)-2 and formula (III)-2, R¹⁰ to R¹³ each independently represent a hydrogen atom or an alkyl group having 1 to 3 carbon atoms. Examples of the alkyl group having 1 to 3 carbon atoms include a methyl group, an ethyl group, an n-propyl group, and an isopropyl group. R¹⁰ to R¹³ each independently represent preferably a hydrogen atom or an alkyl group having 1 to 2 carbon atoms, more preferably a hydrogen atom or a methyl group, and even more preferably a hydrogen atom. In a preferred embodiment, R¹⁰ and R¹¹ each represent a hydrogen atom or an alkyl group having 1 to 3 carbon atoms. In this case, R¹⁰ and R¹¹ each preferably represent a hydrogen atom or an alkyl group having 1 to 2 carbon atoms, more preferably a hydrogen atom or a methyl group, and even more preferably a methyl group. The 2-hydroxycarboxylic acid of formula (II)-2 wherein R¹² represents CH² and R¹³ represents a methyl group is also referred to as 2-hydroxyisobutanoic acid.

In formula (III)-2, n is not particularly limited as long as it is 1 or more, and it is considered that 2-hydroxycarboxylic acid and polymer thereof usually contain multiple types of compounds or polymers with different n. The upper limit of n is not particularly limited, and may be, for example, 30,000 or less, preferably 20,000 or less, more preferably 10,000 or less, 9000 or less, 8000 or less, 7000 or less, 5000 or less, 3,000 or less, 1,000 or less, 500 or less, or the like. The upper limit of n is not particularly limited, but may be, for example, 100 or less, 80 or less, 60 or less, 50 or less, 40 or less, 30 or less, or the like from the viewpoint of easily enhancing the effect of further including a carboxylic acid anhydride. In one preferred embodiment of the present invention, from the viewpoint of production efficiency of the (meth)acrylic acid compound, in the 2-hydroxycarboxylic acid represented by formula (III)-2 and polymer thereof, the ratio of the polymer of the 2-hydroxycarboxylic acid represented by formula (III)-2, preferably n=2 to 30,000, more preferably n=2 to 20,000, even more preferably n=2 to 15,000, and even more preferably 2 to 10,000, to 100 parts by mass of the total of the 2-hydroxycarboxylic acid and polymer thereof is preferably 70 mol % or more, more preferably 80 mol % or more, and even more preferably 90 mol % or more. In addition, from the viewpoint of easily enhancing the effect of further containing a carboxylic acid anhydride, the ratio of the polymer of the 2-hydroxycarboxylic acid represented by formula (III)-2, preferably n=2 to 20, more preferably n=2 to 15, and even more preferably n=2 to 10, to 100 parts by mass of the total of the 2-hydroxycarboxylic acid and its polymer is preferably 70 mol % or more, more preferably 80 mol % or more, and even more preferably 90 mol % or more.

In another embodiment, the production method of the present invention is a method for producing a (meth)acrylic acid compound using a hydroxycarboxylic acid and/or a polymer thereof as a raw material, comprising at least a reaction step of heating a mixture containing a hydroxycarboxylic acid and/or a polymer thereof and a basic catalyst to react with each other to produce a (meth)acrylic acid compound, wherein the weight-average molecular weight of the polymer of the hydroxycarboxylic acid contained in the raw material is 10,000 or more. (2) Next, an embodiment (2) in which the weight-average molecular weight of the polymer of the hydroxycarboxylic acid contained in the raw material is 10,000 or more will be described.

When the weight-average molecular weight of the polymer of the hydroxycarboxylic acid contained in the raw material is 10,000 or more, the (meth)acrylic acid compound can be produced from the hydroxycarboxylic acid and/or the polymer thereof even if the mixture does not further contain the carboxylic acid anhydride, but when the mixture further contains the carboxylic acid anhydride, the (meth)acrylic acid compound can be produced more efficiently.

In this embodiment, the weight-average molecular weight of the polymer of the hydroxycarboxylic acid contained in the raw material is 10,000 or more.

To the extent not inconsistent with this feature, the features relating to the hydroxycarboxylic acid and the polymer thereof described with respect to the embodiment of (1) above apply equally to the embodiment of (2).

The weight-average molecular weight of the polymer of the hydroxycarboxylic acid contained in the raw material is preferably 20,000 or more, more preferably 60,000 or more, still more preferably 70,000 or more, particularly preferably 100,000 or more, and most preferably 130,000 or more from the viewpoint of the production efficiency of the (meth)acrylic acid compound when no carboxylic acid anhydride is added.

The upper limit of the weight average molecular weight of the polymer of the hydroxycarboxylic acid is not particularly limited, but may be, for example, 1,000,000 or less, more preferably 800,000 or less.

Whether the weight-average molecular weight of the polymer of the hydroxycarboxylic acid contained in the raw material is 10,000 or more can be determined, for example, by using the 3-hydroxycarboxylic acid and/or polymer thereof used as the raw material as a measurement sample and measuring the weight-average molecular weight in terms of polystyrene by gel permeation chromatography (GPC). The conditions for measuring the weight average molecular weight may be those described in the Examples.

(Method of Producing Hydroxycarboxylic Acid and/or Polymer Thereof)

The method of producing hydroxycarboxylic acid is not particularly limited, and it can be produced by a known method.

The method of producing 3-hydroxycarboxylic acid polymer is not particularly limited, and for example, it may be chemically synthesized by intermolecular bonding of 3-hydroxycarboxylic acid, or a polymer of 3-hydroxycarboxylic acid produced through a fermentation process can be used. For example, it is possible to use a polymer of 3-hydroxycarboxylic acid produced by a method such as that described in International Publication No. 2011/100608.

The polymer of 3-hydroxycarboxylic acid may be added to the mixture in the production method of the present invention in the form of a composition containing, for example, the polymer of 3-hydroxycarboxylic acid obtained during the production of the polymer of 3-hydroxycarboxylic acid and other by-products, etc.

When a polymerized product of 3-hydroxycarboxylic acid is produced by chemical synthesis, as a dehydration condensation method of 3-hydroxycarboxylic acid, a known method described in Japanese Patent No. 6173314, Journal of Organic Chemistry, 26 (7), pages 2525 to 2528, and the like, and a production method of the following Examples can be used.

When a 3-hydroxycarboxylic acid polymer is produced via a fermentation process, the polymer can be produced by producing 3-hydroxycarboxylic acid by a method described in International Publication No. 2011/100608, International Publication No. 2002/42418, or the like, and then dehydrating and condensing the resulting 3-hydroxycarboxylic acid by the dehydration and condensation method described above. Another method for producing a 3-hydroxycarboxylic acid polymer can be produced using glucose, glycerol, or the like as a raw material, as disclosed in Front. Bioeng. Biotechnol., 9: 646995 (2021).

The method for producing the polymer of 2-hydroxycarboxylic acid is not particularly limited. For example, the polymer may be chemically synthesized by intermolecular bonding of 2-hydroxycarboxylic acid, or a polymer of 2-hydroxycarboxylic acid produced through a fermentation process may be used.

The polymer of 2-hydroxycarboxylic acid may be added to the mixture in the production method of the present invention in the form of a composition containing, for example, the polymer of 2-hydroxycarboxylic acid and other by-products obtained during the production of the polymer of 2-hydroxycarboxylic acid.

When producing the polymer of 2-hydroxycarboxylic acid by chemical synthesis, the method described in International Publication No. 2010/071019 and the like and the production method described in the Examples below can be used as the polymerization via a lactide derivative of 2-hydroxycarboxylic acid.

When a polymer of 2-hydroxycarboxylic acid is produced through a fermentation process, 2-hydroxycarboxylic acid is produced by the method described in Applied and Environmental Microbiology, 2017, 83(3), e02622-16, and the like and the obtained 2-hydroxycarboxylic acid is polymerized through the lactide derivative described above.

The method for producing a polymer of hydroxycarboxylic acid having a weight-average molecular weight of 10,000 or more is not particularly limited, and examples thereof include the method described in the Examples.

### (Basic Catalyst)

The basic catalyst acts as a catalyst when producing a (meth)acrylic acid compound using a hydroxycarboxylic acid and/or polymer thereof as a raw material. The basic catalyst may be a basic substance that acts as a catalyst, and may be a compound that contains a basic site in its molecular structure. The basic catalyst is not particularly limited, and any of those dissolved in the reaction mixture and those not dissolved can be used. The acid dissociation constant pKa of the basic catalyst is preferably 0.5 to 40, more preferably 1 to 30, even more preferably 2 to 15, and particularly preferably 3 to 7. When the pKa is equal to or greater than a certain value, the reaction efficiency is improved, and when the pKa is equal to or less than a certain value, the occurrence of unintended reactions (side reactions) can be sufficiently suppressed. The pKa of the basic catalyst in the present invention refers to the pKa of the conjugate acid, and can be determined by acid-base titration on the conjugate acid, but the pKa listed in Tables 10 and 19 on pages II-317 to II-321 of the Basic Chemistry Handbook, Revised 4th Edition may also be used. Specific examples of the basic catalyst include carboxylates, amines, inorganic hydroxides, ammonium hydroxides, metal amides, metal alkoxides, and the like, and preferably carboxylates. More specifically, examples of the carboxylic acid alkali metal salts include alkali metal salts of α,β-ethylenically unsaturated carboxylic acids and alkali metal salts of aliphatic carboxylic acids, quaternary ammonium salts of carboxylic acids (quaternary ammonium salts of α,β-ethylenically unsaturated carboxylic acids and quaternary ammonium salts of aliphatic carboxylic acids), ammonium betaine, alkali metal hydroxides (e.g., potassium hydroxide, sodium hydroxide, lithium hydroxide, etc.), alkaline earth metal hydroxides (magnesium hydroxide, calcium hydroxide, etc.), transition metal hydroxides (copper hydroxide, etc.), alkali metal carbonates (potassium carbonate, sodium carbonate, lithium carbonate, etc.), alkaline earth metal carbonates (magnesium carbonate, calcium carbonate, etc.), Examples of the organic solvent include magnesium, calcium carbonate, barium carbonate, etc.), alkali metal hydrogen carbonates (potassium hydrogen carbonate, sodium hydrogen carbonate, lithium hydrogen carbonate, etc.), alkaline earth metal hydrogen carbonates (magnesium hydrogen carbonate, calcium hydrogen carbonate, barium hydrogen carbonate, etc.), alkoxides (for example, alkali metal alkoxides such as potassium methoxide and sodium methoxide), alkali metal amides (potassium amide, sodium amide, etc.), amines or ammoniums (alkylamines such as triethylamine; tertiary arylamines such as N,N-dimethylaniline), heterocyclic amines such as pyridine, and quaternary ammonium hydroxides.

In a preferred embodiment, the basic catalyst may be preferably a carbonate or a hydrogen carbonate, and more preferably at least one selected from the group consisting of alkali metal carbonates, alkaline earth metal carbonates, alkali metal hydrogen carbonates, and alkaline earth metal hydrogen carbonates. More specifically, the above-mentioned can be mentioned.

When a carbonate or a hydrogen carbonate is used as the alkali metal salt, as will be described later, the carbonate ion or hydrogen carbonate ion disappears from the reaction system as carbon dioxide during the reaction, while the remaining alkali metal ion becomes the alkali metal salt of the desired α,β-ethylenically unsaturated carboxylic acid and is considered to act as a catalyst.

In a preferred embodiment, the basic catalyst may be preferably an alkali metal carboxylate, and more preferably at least one selected from the group consisting of alkali metal salts of α,β-ethylenically unsaturated carboxylic acids and alkali metal salts of aliphatic carboxylic acids, and even more preferably at least one selected from the group consisting of alkali metal salts of α,β-ethylenically unsaturated monocarboxylic acids and alkali metal salts of aliphatic monocarboxylic acids. Examples of the alkali metal salts of α,β-ethylenically unsaturated carboxylic acids include alkali metal salts of (meth)acrylic acid. Examples of the alkali metal include lithium, sodium, potassium, etc., and preferably sodium or potassium. More specifically, examples include potassium (meth)acrylate and sodium (meth)acrylate. Examples of the alkali metal salts of aliphatic carboxylic acids include potassium acetate and sodium acetate.

When an alkali metal salt of an α,β-ethylenically unsaturated monocarboxylic acid is used as the alkali metal salt of the carboxylate, an α,β-ethylenically unsaturated monocarboxylic acid derived from the alkali metal salt of the α,β-ethylenically unsaturated monocarboxylic acid is generated. Therefore, if the structure of the α,β-ethylenically unsaturated monocarboxylic acid in the alkali metal salt of the α,β-ethylenically unsaturated monocarboxylic acid is the same as that of the (meth)acrylic acid compound obtained by the production method of the present invention, the amount of impurities that may be contained in the (meth)acrylic acid compound obtained by the production method of the present invention can be reduced.

When an alkali metal salt of an aliphatic carboxylic acid, preferably an alkali metal salt of an aliphatic monocarboxylic acid, more preferably an alkali metal salt of an aliphatic monocarboxylic acid, is used as the alkali metal salt of the carboxylate, an aliphatic (mono)carboxylic acid is generated as a by-product. Since the aliphatic (mono)carboxylic acid is easily removed by distillation, washing with water, or the like, the amount of impurities that may be contained in the (meth)acrylic acid compound obtained by the production method of the present invention can be greatly reduced. Therefore, it is preferable to use, as the basic catalyst, at least one selected from the group consisting of an alkali metal salt of an α,β-ethylenically unsaturated monocarboxylic acid and an alkali metal salt of an aliphatic carboxylic acid, and more preferably at least one selected from the group consisting of an alkali metal salt of (meth)acrylic acid and an alkali metal salt of an aliphatic carboxylic acid, since the amount of impurities that may be contained in the (meth)acrylic acid compound obtained by the production method of the present invention can be greatly reduced.

Therefore, it is preferable to use at least one selected from the group consisting of alkali metal salts of α,β-ethylenically unsaturated monocarboxylic acids and alkali metal salts of aliphatic carboxylic acids as the basic catalyst, and more preferably to use at least one selected from the group consisting of alkali metal salts of (meth)acrylic acid and alkali metal salts of aliphatic carboxylic acids as the basic catalyst, since this makes it possible to greatly reduce the amount of impurities that may be contained in the (meth)acrylic acid compound obtained by the production method of the present invention.

In another preferred embodiment, the basic catalyst may be preferably a quaternary ammonium salt of a carboxylic acid, more preferably at least one selected from the group consisting of a quaternary ammonium salt of an α,β-ethylenically unsaturated carboxylic acid and a quaternary ammonium salt of an aliphatic carboxylic acid, and even more preferably at least one selected from the group consisting of a quaternary ammonium salt of an α,β-ethylenically unsaturated monocarboxylic acid and a quaternary ammonium salt of an aliphatic monocarboxylic acid. Examples of the quaternary ammonium salt of an α,β-ethylenically unsaturated carboxylic acid include a quaternary ammonium salt of (meth)acrylic acid. Examples of the ammonium ion of such a quaternary ammonium salt include an ammonium ion represented by N⁺(—R^{a})₄[R^{a} each independently represent a hydrocarbon group which may have a substituent (for example, an aromatic substituent such as a benzyl group), for example, an alkyl group having 1 to 12 carbon atoms which may have a substituent]. Specific examples of the quaternary ammonium carboxylate include tetraethylammonium (meth)acrylate, tetrabutylammonium (meth)acrylate, benzyltributylammonium (meth)acrylate, tetraethylammonium aliphatic carboxylate, tetrabutylammonium aliphatic carboxylate, and benzyltributylammonium aliphatic carboxylate.

In another preferred embodiment, the basic catalyst may be ammonium betaine. Examples of ammonium betaine include trimethylglycine, triethylglycine, tributylglycine, N-lauryl-N,N-dimethylglycine, N-(meth)acryloyloxyethyl-N,N-dimethylammonium-α-N-methylcarboxybetaine, and vinyl copolymers containing N-(meth)acryloyloxyethyl-N,N-dimethylammonium-α-N-methylcarboxybetaine.

### (Carboxylic Acid Anhydride)

According to one embodiment of the manufacturing method of the present invention, the mixture containing a hydroxycarboxylic acid and/or polymer thereof and a basic catalyst further contains a carboxylic acid anhydride. When the weight-average molecular weight of the polymer of the hydroxycarboxylic acid contained in the raw material is 10,000 or more, the carboxylic acid anhydride is not an essential component and may or may not be contained in the mixture.

The carboxylic acid anhydrides are compounds formed by dehydration condensation of two carboxylic acids, and are represented by the general formula: R^{b}—CO—O—CO—R^{b′}[R^{b} and R^{b′} each independently represent a monovalent organic group]. In the manufacturing method of the present invention, it is considered that the presence of a carboxylic acid anhydride and a basic catalyst cause a reaction to proceed that produces a (meth)acrylic acid compound using a hydroxycarboxylic acid and/or polymer thereof as a raw material. The carboxylic acid anhydride is not particularly limited, and examples include (meth)acrylic acid anhydride, acetic acid anhydride, propionic acid anhydride, oxalic acid anhydride, succinic acid anhydride, maleic acid anhydride, phthalic acid anhydride, and benzoic acid anhydride. In addition, one type of carboxylic acid anhydride may be used, or two or more types of carboxylic acid anhydrides may be used. Among these, from the viewpoint of ease of availability and efficient manufacturing of (meth)acrylic acid compounds, it is preferable to use (meth)acrylic acid anhydride, and it is more preferable to use (meth)acrylic acid anhydride having the structure described below.

In a preferred embodiment using (meth)acrylic anhydride as the carboxylic acid anhydride, the (meth)acrylic anhydride is preferably a compound obtained by dehydration condensation of two compounds having a (meth)acrylic acid skeleton and a carboxylic acid skeleton. In this embodiment, the (meth)acrylic anhydride is preferably a compound represented by formula (I):

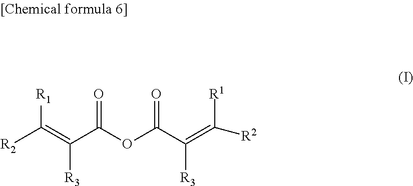

Wherein
R¹ to R³ each independently represent a hydrogen atom or an alkyl group having 1 to 3 carbon atoms.

In this specification, the (meth)acrylic anhydride represented by formula (I) is also referred to as “(meth)acrylic anhydride (I)”. In a preferred embodiment of the production method of the present invention, when (meth)acrylic anhydride (I) and a basic catalyst are present, it is considered that the reaction of producing a (meth)acrylic acid compound using a hydroxycarboxylic acid and/or polymer thereof as a raw material is more likely to proceed.

R¹ to R³ in formula (I) each independently represent a hydrogen atom or an alkyl group having 1 to 3 carbon atoms. Examples of the alkyl group having 1 to 3 carbon atoms include a methyl group, an ethyl group, an n-propyl group, and an isopropyl group. It is preferable that R¹ to R³ have a structure corresponding to the acrylic acid compound to be produced, from the viewpoints of isolability of the obtained acrylic acid compound and reduction of impurities that may be contained in the compound. For example, when the 3-hydroxycarboxylic acid and/or polymer thereof used as the raw material is represented by the above formula (III), it is preferable that R¹ and R² in formula (I) are the same as R⁴ and R⁵ in formula (III), respectively, and R³ in formula (I) is preferably the same as R⁶ in formula (III). R¹ to R³ each independently preferably represent a hydrogen atom or an alkyl group having 1 to 2 carbon atoms, more preferably a hydrogen atom or a methyl group, and even more preferably a hydrogen atom. In a preferred embodiment of the present invention, R² represents a hydrogen atom or an alkyl group having 1 to 3 carbon atoms, and R¹ and R³ represent a hydrogen atom. In this case, R² preferably represents a hydrogen atom or an alkyl group having 1 to 2 carbon atoms, more preferably a hydrogen atom or a methyl group, and further preferably a hydrogen atom.

### (Other Components)

The mixture containing the hydroxycarboxylic acid and/or polymer thereof and the basic catalyst may contain one or more other components. Examples of the other components include additives such as polymerization inhibitors, solvents, etc.

Polymerization inhibitor is not particularly limited, but examples of phenolic compounds include hydroquinone, p-methoxyphenol, cresol, t-butylcatechol, 3,5-di-t-butyl-4-hydroxytoluene, 2,2′-methylenebis(4-methyl-6-t-butylphenol), 2,2′-methylenebis(4-ethyl-6-butylphenol), 4,4′-thiobis(3-methyl-6-t-butylphenol), etc., and examples of nitrogen-containing compounds include nitroxides such as piperidine-1-oxyl, pyrrolidine-1-oxyl, 2,2,6,6-tetramethyl-4-oxopiperidine-1-oxyl, and 2,2,6,6-tetramethylpiperidine-1-oxyl, phenothiazine, 3,7-dioctylphenothiazine, 3,7-Examples of phenothiazine derivatives such as dicumylphenothiazine and 3,7-di-t-butylphenothiazine, and transition metal salts include copper salts such as copper dialkyldithiocarbamate (wherein the alkyl group is any of methyl, ethyl, propyl, and butyl groups and may be the same or different), copper acetate, copper salicylate, copper thiocyanate, copper nitrate, copper chloride, copper carbonate, copper hydroxide, and copper acrylate; manganese dialkyldithiocarbamate (wherein the alkyl group is any of methyl, ethyl, propyl, and butyl groups and may be the same or different), manganese diphenyldithiocarbamate, manganese formate, manganese acetate, manganese octanoate, manganese naphthenate, manganese permanganate, and manganese salt of ethylenediaminetetraacetic acid. When a polymerization inhibitor is used, one type of polymerization inhibitor may be used, or two or more types may be used in combination. When the mixture contains a polymerization inhibitor, the polymerization of the (meth)acrylic acid compound generated in the reaction step can be suppressed, which is preferable since it is possible to improve the production efficiency of the (meth)acrylic acid compound. In addition, it is also possible to suppress the polymerization of by-products that may be generated. The polymerization inhibitor is preferably a nitroxide such as piperidine-1-oxyl, pyrrolidine-1-oxyl, 2,2,6,6-tetramethyl-4-oxopiperidine-1-oxyl, or 2,2,6,6-tetramethylpiperidine-1-oxyl, or a phenothiazine derivative such as phenothiazine, 3,7-dioctylphenothiazine, 3,7-dicumylphenothiazine, or 3,7-di-t-butylphenothiazine, and more preferably 2,2,6,6-tetramethyl-4-oxopiperidine-1-oxyl or 2,2,6,6-tetramethylpiperidine-1-oxyl. When the mixture contains a polymerization inhibitor, the content of the polymerization inhibitor is preferably 0.005 to 5 parts by mass, more preferably 0.01 to 2 parts by mass, and even more preferably 0.05 to 0.5 parts by mass, based on 100 parts by mass of the polymer of 3-hydroxycarboxylic acid. When the amount of the polymerization inhibitor is equal to or more than the above lower limit, the polymerization inhibitory effect of the (meth)acrylic acid compound can be enhanced.

The mixture in the production method of the present invention may or may not contain a solvent. As the solvent that may be contained in the mixture, an aprotic organic solvent is preferable from the viewpoint of less inhibiting the production of the (meth)acrylic acid compound. Examples of such an aprotic organic solvent include ethyl acetate, tetrahydrofuran, toluene, methyl ethyl ketone, and cyclopentanone. The content of the solvent in the mixture is not particularly limited, and may be, for example, 0 to 80 mass % based on the total amount of the mixture.

In a preferred embodiment of the present invention, from the viewpoint of isolability of the (meth)acrylic acid compound to be produced, the content of the solvent contained in the mixture, in particular, the content of the solvent having a boiling point lower than the boiling point of the (meth)acrylic acid compound to be produced, is preferably 0 to 50 mass %, more preferably 0 to 30 mass %, even more preferably 0 to 20 mass %, even more preferably 0 to 10 mass %, and particularly preferably 0 to 5 mass %, based on the total amount of the mixture.

In the mixture containing a hydroxycarboxylic acid and/or polymer thereof and a basic catalyst, or a mixture containing a hydroxycarboxylic acid and/or polymer thereof, a basic catalyst, and a carboxylic anhydride, which are reacted in the production method of the present invention, the amount of the basic catalyst relative to a total of 100 parts by mass of the hydroxycarboxylic acid and polymer thereof is preferably 0.01 to 20 parts by mass, more preferably 0.1 to 10 parts by mass, and even more preferably 0.5 to 5 parts by mass, from the viewpoint of improving the yield of (meth)acrylic acid compounds.

In the mixture containing a hydroxycarboxylic acid and/or polymer thereof, a basic catalyst, and a carboxylic anhydride, the amount of the carboxylic anhydride represented by formula (I) relative to a total of 100 parts by mass of the hydroxycarboxylic acid and polymer thereof is preferably 0.1 to 50 parts by mass, more preferably 1 to 30 parts by mass, and even more preferably 5 to 20 parts by mass, from the viewpoint of improving the yield of (meth)acrylic acid compounds.

In the mixture, the amount of the carboxylic acid anhydride represented by formula (I) relative to the total amount of the hydroxycarboxylic acid and polymer thereof is preferably 0.05 to 25 mol %, more preferably 0.5 to 15 mol %, and even more preferably 2.5 to 10 mol %, from the viewpoint of improving the yield of the (meth)acrylic acid compound.

### (Method of Producing (Meth)Acrylic Acid Compounds)

The method for producing a (meth)acrylic acid compound, comprising at least a reaction step of heating a mixture containing a hydroxycarboxylic acid and/or a polymer thereof and a basic catalyst to react with each other to produce a (meth)acrylic acid compound, wherein (1) the mixture further contains a carboxylic acid anhydride, and/or (2) the weight-average molecular weight of the polymer of the hydroxycarboxylic acid contained in the raw material is 10,000 or more. The (meth)acrylic acid compound produced by the method of the present invention is not particularly limited as long as it is a compound having a (meth)acrylic acid skeleton, and may be acrylic acid, methacrylic acid, and compounds in which at least one hydrogen atom of these is substituted with an alkyl group having 1 to 3 carbon atoms, as well as derivatives thereof (for example, salts and esters). The (meth)acrylic acid compound produced by the method of the present invention usually has a structure corresponding to the polymer of the hydroxycarboxylic acid used as the raw material. For example, when a hydroxycarboxylic acid and polymer thereof are represented by formula (III)-1 or formula (III)-2, the (meth)acrylic acid compound obtained by the manufacturing method of the present invention is considered to be a compound represented by formula (IV).

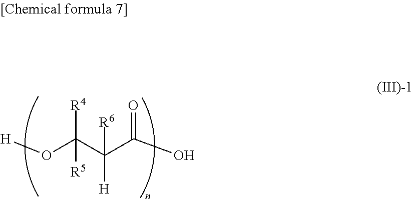

wherein
R⁴ to R⁶ each independently represent a hydrogen atom or an alkyl group having 1 to 3 carbon atoms, and n represents an integer of 1 or more,

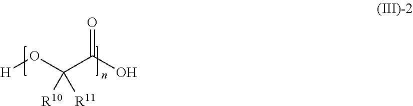

wherein
R¹⁰ and R¹¹ each independently represent a hydrogen atom or an alkyl group having 1 to 3 carbon atoms, and n represents an integer of 1 or more,
the (meth)acrylic acid compound obtained by the manufacturing method of the present invention is considered to be a compound represented by formula (IV):

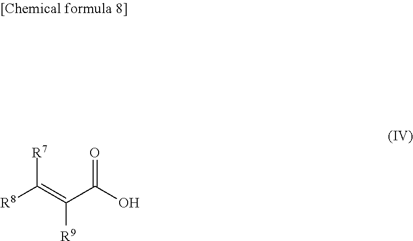

Wherein
R⁷ to R⁹ independently represent a hydrogen atom or an alkyl group having 1 to 3 carbon atoms.

It is considered that R⁷ and R⁹ in formula (IV) correspond to R⁴ and R⁵ in formula (III)-1, respectively, and R⁹ corresponds to R⁶. It is also considered that R⁹ in formula (IV) corresponds to R¹⁰ in formula (III)-2, and R⁷ and R⁸ correspond to R¹¹. The (meth)acrylic acid compound obtained by the production method of the present invention may be one type of (meth)acrylic acid compound or a mixture of two or more types of (meth)acrylic acid compounds.

As long as the manufacturing method of the present invention includes the above reaction steps, there are no limitations on the reaction mechanism, but it is considered that the reaction steps proceed, for example, according to the following reaction mechanism. (1) The case where the mixture further includes a carboxylic acid anhydride and/or (2) The case where the weight-average molecular weight of the polymer of hydroxycarboxylic acid included in the raw material is 10,000 or more will be described. First, the case (1) where the mixture further includes a carboxylic acid anhydride will be described.

In the following reaction mechanism, the case where the hydroxycarboxylic acid is 3-hydroxypropionic acid which is a kind of 3-hydroxycarboxylic acid, the basic catalyst is sodium acrylate, and the carboxylic acid anhydride is acrylic acid anhydride will be described as an example, however, this does not mean that the components are limited to these, and the mechanism is similarly applicable to other 3-hydroxycarboxylic acids and/or polymers thereof, other basic catalysts, and other carboxylic anhydrides. Furthermore, the production method of the present invention is not limited to the following reaction mechanism in any way.

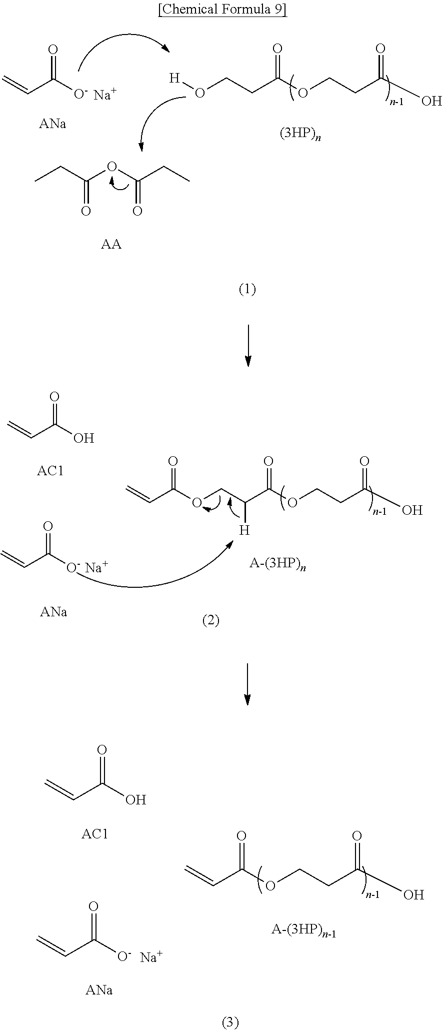

In the above reaction scheme, ANa is sodium acrylate, (3HP)ₙ is 3-hydroxypropionic acid or polymer thereof, AA is acrylic anhydride, and AC is acrylic acid. First, as shown in (1), the negative charge A⁻ of ANa acts on the proton of the terminal hydroxyl group of (3HP)ₙ, and the negative charge generated on the oxygen atom of (31HP)ₙ attacks the carbonyl carbon of AA, AC1, ANa, and A-(3HP)ₙ shown in (2) are generated. Note that in (1), the chemical formula of (3HP)ₙ is shown as 3HP-(3HP)ₙ₋₁. Next, as shown in (2), the negative charge A⁻ of ANa acts on A-(3HP)ₙ, AC2, ANa, and A-(3HP)ₙ₋₁ shown in (3) are generated. Next, not shown in the above reaction scheme, the negative charge A⁻ of ANa acts on A-(3HP)ₙ₋₁ in the same manner as in the reaction shown in (2), and AC3, ANa, and A-(3HP)ₙ₋₂ are generated. As this reaction continues, it is considered that n in (3HP)ₙ decreases by one each, and acrylic acid is generated sequentially. In addition, in (1) in the above reaction scheme, one hydroxyl group of (3HP)ₙ reacts with AA, but even if the hydroxyl groups at both ends of (3HP)ₙ are reacted with AA to become A-(3HP)_{n-A}, the reaction is considered to proceed in the same manner. In addition, in the above reaction scheme, it is considered that the synergistic effect of the presence of AA and ANa allows the above reaction to proceed under milder conditions than when the reaction is carried out using only ANa.

In the above reaction, it is considered that a reverse reaction may occur in which A-(3HP)ₙ₋₁ shown in (3) further undergoes a Michael addition reaction with AC2 or ANa to return to A-(3HP)ₙ. For example, it is considered that it is possible to suppress such a reverse reaction by removing the resulting AC2 from the reaction system.

The basic compound (for example, ANa) reacted in (1) may be a catalytic amount, and ANa reacted in (2) and (3) and the like is generated sequentially in the system. Therefore, it is understood that the basic compound used in the manufacturing method of the present invention is not particularly limited as long as it is a basic compound that acts on the proton of the terminal hydroxyl group of (3HP)ₙ, and that those having various reactivities may be used. It is also understood that the (meth)acrylic anhydride may be, for example, a compound represented by formula (I), and the structure thereof is not particularly limited, but it is understood that by using a (meth)acrylic anhydride corresponding to the polymer of the 3-hydroxycarboxylic acid used, the acrylic acid compound obtained as the product can be a single compound, and impurities can be further reduced.

Next, an example will be described in which the hydroxycarboxylic acid is 2-hydroxyisobutyric acid, which is a kind of 2-hydroxycarboxylic acid, the basic catalyst is sodium methacrylate, and the carboxylic anhydride is methacrylic anhydride. As with the above, this does not mean that the components are limited to these, and the mechanism also applies to other 2-hydroxycarboxylic acids and/or polymer thereof, other basic catalysts, and other carboxylic anhydrides.

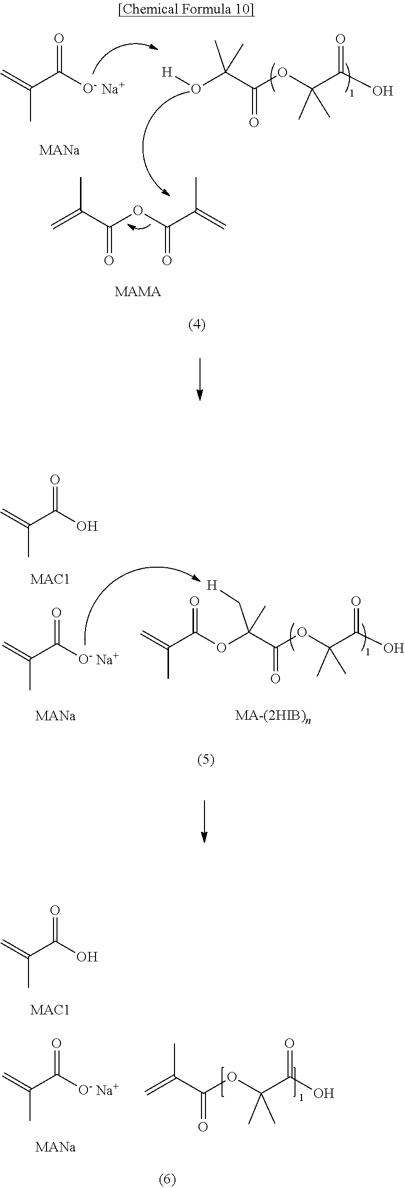

In the above reaction scheme, MANa is sodium methacrylate, (2HIB)ₙ is 2-hydroxyisobutyric acid or polymer thereof, MAMA is methacrylic anhydride, and MAC is methacrylic acid. First, as shown in (4), the negative charge MA⁻ of MANa acts on the proton of the terminal hydroxyl group of (2HIB)ₙ, and the negative charge generated on the oxygen atom of (2HIB)ₙ attacks the carbonyl carbon of MAMA, MAC1, MANa, and MA-(2HIB)ₙ, as shown in (5) are generated. Note that in (4), the chemical formula of (2HIB)ₙ is shown as 2HIB-(2HIB)ₙ₋₁. Next, as shown in (5), the negative charge MA⁻ of MANa acts on MA-(2HIB)ₙ, MAC2, MANa, and MA-(2HIB)ₙ₋₁, as shown in (6) are generated. Next, although not shown in the above reaction scheme, the negative charge MA⁻ of MANa acts on MA-(2HIB)ₙ₋₁, similar to the reaction shown in (5), MAC3, MANa, and MA-(2HIB)ₙ₋₂ are generated. As this reaction continues, it is considered that n in (2HIB)ₙ decrease by one each, and methacrylic acid is sequentially generated. In addition, in (4) in the above reaction scheme, one hydroxyl group of (2HIB)ₙ reacts with MAMA, but even if the hydroxyl groups at both ends of (2HIB)ⁿ react with MAMA to become MA-(2HIB)ₙ-MA, it is considered that the reaction will proceed in the same manner. In addition, in the above reaction scheme, it is considered that the synergistic effect of the presence of MAMA and MANa allows the above reaction to proceed under milder conditions than when the reaction is carried out using only MANa.

Here, it was found that when the weight-average molecular weight of the polymer of hydroxycarboxylic acid contained in the raw material is 10,000 or more, a (meth)acrylic acid compound can be obtained when the carboxylic acid anhydride is not present. The reason for this is not clear, but it is considered that the reaction proceeds, for example, according to the following mechanism. Note that the following reaction mechanism will be explained using specific compounds as examples, but this does not mean that the components are limited to these. Furthermore, the production method of the present invention is not limited to the following reaction mechanism.

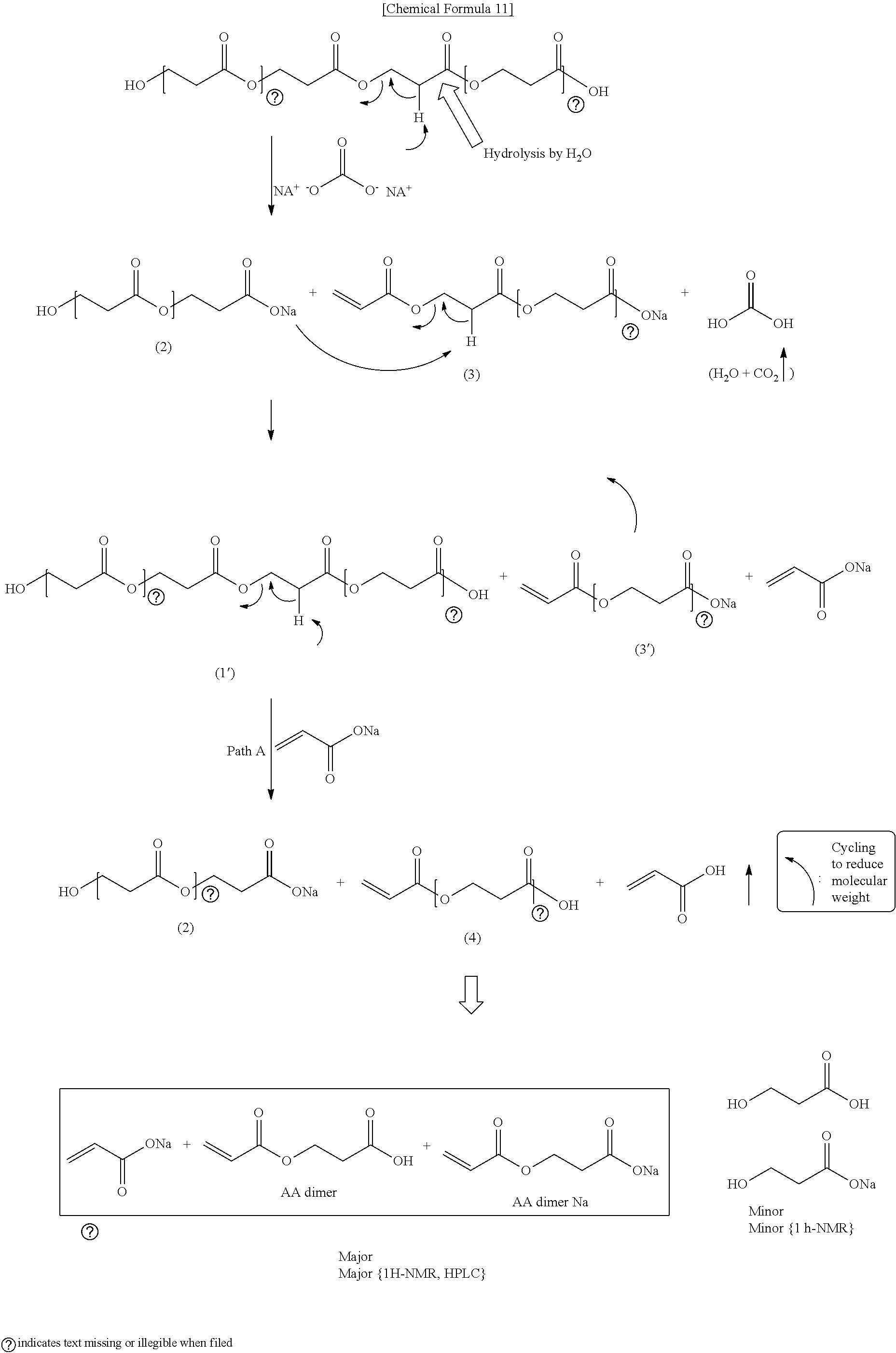

It is considered that when a polymer of 3-hydroxycarboxylic acid having a weight-average molecular weight of 10,000 or more represented by formula (1) reacts with a basic catalyst (sodium carbonate in the reaction scheme), compounds represented by formula (2) and formula (3) are generated. On the other hand, when acrylic anhydride is not used, the polymer of formula (2′) and (4) will ultimately remain as the main component (Major) or the minor component (Minor) For this reason, for example, when a polymer of 3-hydroxycarboxylic acid having a weight average molecular weight of 1000 is compared with a polymer of 3-hydroxycarboxylic acid having a weight average molecular weight of 10,000, the number of molecules (moles) of the latter is 1/10 of the number of molecules of the former when the weight is the same. Therefore, in the polymer of 3-hydroxycarboxylic acid having a weight-average molecular weight of 10,000, the residue as the main component (Major) and the minor component (Minor) is considered to be 1/10 of the polymerized product of 3-hydroxycarboxylic acid having a weight-average molecular weight of 1000, and the yield of acrylic acid is increased. That is, the lower the molecular weight, the more the residues as the main component (Major) and the minor component (Minor) will increase, and the yield of acrylic acid will decrease. At this time, when the water content is large, the polymer represented by formula (1) will be hydrolyzed, the weight-average molecular weight will decrease, and the molecular weight will decrease, so that it is considered that the residues as the main component (Major) and the trace component (Minor) will increase and the yield of acrylic acid will decrease.

In the production method of the present invention, the water content in the mixture containing the hydroxycarboxylic acid and/or polymer thereof and the basic catalyst is preferably low. For example, the water content is preferably 0 to 15 mass %, more preferably 0 to 10 mass %, and even more preferably 0 to 5 mass % based on the total amount of the mixture. When the water content is equal to or less than the above upper limit, the amount of water contained in the (meth)acrylic acid compound product can be reduced, improving the handleability of the product. The amount of water contained in the (meth)acrylic acid compound product is preferably 0.1 mass % or less based on the total amount of the (meth)acrylic acid compound. In addition, when the weight-average molecular weight of the polymer of the hydroxycarboxylic acid contained in the raw material is 10,000 or more, it is considered that when the amount of water is equal to or less than the above upper limit, the residue as the main component (Major) and the trace component (Minor) in the above-described reaction scheme can be further reduced.

The production method of the present invention includes at least a reaction step wherein a mixture containing at least a hydroxycarboxylic acid and/or polymer thereof with a basic catalyst is heated to react with each other to produce a (meth)acrylic acid compound.

The reaction step may be carried out, for example, by heating the mixture placed in a reaction vessel. The reaction step may be carried out in a batch process or continuous process. Since the (meth)acrylic acid generated in the reaction step is easily polymerized, it is preferable to carry out a step of distilling off the (meth)acrylic acid compound generated in the reaction step from the viewpoint of preventing such polymerization and improving the yield of the reaction product. For example, it is more preferable that the reaction step and the distillation step of (meth)acrylic acid to be produced are performed simultaneously. Examples of reaction apparatus include a reactor equipped with a stirrer, and a reaction apparatus configured to be capable of both heating the mixture and distilling off the produced (meth)acrylic acid (for example, an apparatus equipped with a thin film distiller or a Kugelrohr).

The heating condition is not particularly limited as long as the hydroxycarboxylic acid and/or polymer thereof reacts with the basic catalyst, preferably together with the carboxylic anhydride, to produce a (meth)acrylic acid compound, but from the viewpoint of reaction efficiency, the heating temperature is preferably 50 °C or higher, more preferably 70 °C or higher, even more preferably 80 °C or higher, and even more preferably 90 °C or higher. From the viewpoint of easily suppressing unnecessary side reactions, the heating temperature may be preferably less than 300 °C, more preferably 250 °C or lower, even more preferably 220 °C or lower, even more preferably 200 °C or lower, particularly preferably 180 °C or lower, particularly preferably 160 °C or lower, and extremely preferably 140 °C or lower. In conventional manufacturing methods for producing (meth)acrylic acid compounds from polymers of hydroxycarboxylic acids, the heating temperature was as high as more than 300 °C, but the production method of the present invention makes it possible to produce (meth)acrylic acid compounds under milder conditions. The heating temperature in the reaction step may be preferably in the range of 50 to 300 °C, 70 to 250 °C, 80 to 220 °C, 80 to 200 °C, 80 to 180 °C, 90 to 160 °C, 90 to 140 °C, and the like. In a preferred embodiment of the present invention, from the viewpoint of carrying out the reaction under milder conditions compared to the prior art, the heating temperature in the reaction step is preferably 80 to 220 °C, more preferably 80 to 200 °C, even more preferably 80 to 180 °C, even more preferably 90 to 160 °C, and particularly preferably 90 to 140 °C.

The heating time is not particularly limited and may be adjusted as appropriate depending on the progress of the reaction. When the reaction process is carried out in a continuous process, the raw material hydroxycarboxylic acid and/or polymer thereof is added to the reaction system while heating, and the (meth)acrylic acid compound produced is removed from the reaction system, so that the reaction process can be carried out continuously. When the carboxylic acid anhydride is used, it is preferable to also add the carboxylic acid anhydride, which is considered to be consumed by the reaction, to the reaction system. In this case, the heating time is also not particularly limited.

In a preferred embodiment of the present invention, the production method of the present invention further comprises a step of distilling off the (meth)acrylic acid compound produced in the reaction step. By distilling off the produced (meth)acrylic acid compound from the reaction system, it becomes possible to shift the equilibrium of the reaction in the direction of producing the (meth)acrylic acid compound, and as a result, the yield of the (meth)acrylic acid compound can be improved. In other words, it is preferable that the step of distilling off the (meth)acrylic acid compound is carried out simultaneously with the reaction step.

Methods for distilling off the (meth)acrylic acid compound include distillation, particularly vacuum distillation. The pressure (degree of vacuum) in the system in the vacuum distillation is preferably lower than the vapor pressure of the resulting (meth)acrylic acid compound. Therefore, the upper limit of the degree of reduced pressure is preferably 101 kPa or less, more preferably 67 kPa or less, and even more preferably 40 kPa or less. In addition, from the viewpoint of making high-boiling components such as polymers (particularly dimers) of the (meth)acrylic acid compound less likely to be contained in the distillate, the lower limit of the degree of reduced pressure is preferably 1.3 kPa or more, preferably 6.7 kPa or more, more preferably 11 kPa or more, and even more preferably 16 kPa or more.

According to a preferred embodiment of the present invention, the production method of the present invention can be produced a (meth)acrylic acid compound with few impurities. For example, the (meth)acrylic acid compound produced by the production method of the present invention has a low content of by-products such as water and propionic acid.

According to the production method of the present invention, a novel method for producing a (meth)acrylic acid compound can be provided. In a preferred embodiment of the present invention, the (meth)acrylic acid compound produced by the production method of the present invention has few impurities and can be used as a useful raw material in the production of resins such as poly(meth)acrylic acid.

### EXAMPLES

### Examples

The present invention will be described in more detail below with reference to examples, but the present invention is not limited to the following examples. Unless otherwise specified, “%” and “parts” in the following description mean “% by mass” and “parts by mass”, respectively.

### [HPLC]

The measurement conditions of HPLC are: column; YMC-pack ODS-AM-302 150 mm×φ4.6 mm S-5 m, 120 A; temperature 40 °C; flow rate 1 mL/min; detection wavelength 210 nm; mobile phase; acetonitrile/0.5 wt % phosphoric acid aqueous solution (ratio 1/1); analysis time: 25 min.

### [¹H-NMR]

The measurement conditions of ¹H-NMR are: 500 MHz, 25 °C, solvent; deuterated chloroform, internal standard; tetramethylsilane.

### [GC]

The measurement conditions of GC are: Column: Capillary column 007-1701, length 25 m, inner diameter 0.32 mm, liquid phase film thickness 1 m (manufactured by Quadex), Column temperature: 50 °C (3 min) to Heating 10 °C/min to 100 °C (0 min) to Heating 15 °C/min to 250 °C (15 min), Injection temperature: 220 °C, Detector (FID) temperature: 220 °C, Carrier gas: He, Flow rate: 2.23 mL/min (inlet pressure 85.0 kPa), Split ratio: 50.0, Analysis time 25 min.

### [GPC]

The measurement conditions of The GPC are: column; TSK-gel SuperAWH-H (4 columns) (manufactured by Tosoh Corporation), mobile phase; N,N-dimethylformamide (0.03 M lithium bromide solution), thermostatic bath; 40 °C, flow rate; 0.6 mL/min, analysis time; 30 min. Mw is the value converted to standard polystyrene.

### Preparation Example 1: Synthesis of Raw Material (A) Containing 3-Hydroxypropionic Acid and Polymer Thereof

To 10 g (33.3 mmol) of a 30% by mass aqueous solution of 3-hydroxypropionic acid (hereinafter, 3-HPA) (manufactured by Tokyo Chemical Industry Co., Ltd.), 6.38 g (33.3 mmol) of EDC·HCl (1-ethyl-3-(3-dimethylaminopropyl)carbodiimide hydrochloride) (manufactured by Tokyo Chemical Industry Co., Ltd.) divided into four portions under water cooling were added at 30 °C or lower, and the mixture was stirred at 40 °C for 4 hours. After confirming the disappearance of 3-HPA by HPLC, an extraction operation was carried out four times by adding 4 times the amount of ethyl acetate to the reaction solution to obtain an extract. The extract was concentrated under reduced pressure using an evaporator in a water bath at 55 °C to obtain 1.85 g of raw material (A) containing 3-HPA and/or polymer thereof as a colorless, transparent oil. Analysis of raw material (A) by HPLC revealed that the 3-HPA polymer included dimers to decamers (n=2-10), with the proportion of 3-HPA polymers (n=2-10) in raw material (A) being 94% by mass, and the proportion of 3-HPA being 6% by mass. The result of the HPLC analysis is shown in FIG. 1.

Furthermore, the raw material (A)'s average of molecular weight was calculated from the proton ratio of ¹H-NMR, and it was found to be a trimer. The molecular weight of the 3-HPA polymer closest to this average value is 234 (trimer), so hereafter raw material (A) will also be described as a trimer with a weight-average molecular weight (Mw) of 234.

### Preparation Example 2: Synthesis of Raw Material (B) Containing 3-HPA and Polymer Thereof

Instead of 10 g of 30% by mass 3-hydroxypropionic acid aqueous solution, 5 g (21.3 mmol) of raw material (A) obtained in Preparation Example 1 and 1.00 g of distilled water were used, and the amount of EDC·HCl was changed to 5.32 g (27.8 mmol), in the same manner was followed as in Preparation Example 1 to obtain 2.32 g of raw material (B) containing 3-HPA and polymer thereof as a colorless, transparent oil. HPLC analysis of raw material (B) showed that raw material (B) contained 95% by mass of 3-HPA polymer, and that the 3-HPA polymer included tetramers to 15 mers (n=4 to 15). The percentage of each polymer in the 3-HPA polymers (n=4 to 15) was as follows: tetramer: 29% by mass, hexamer: 24% by mass, total of octamers and nonamers: 31% by mass, total of decamers and above: 16% by mass.

Furthermore, as with raw material (A) above, the average molecular weight of raw material (B) was calculated from the HPLC area ratio, and the result was a 6.5 mer, meaning that raw material (B) is a heptamer with a weight-average molecular weight of 522.

### Preparation Example 3: Synthesis of Raw Material (C) Containing 3-HPA Polymer

1.20 g (16.65 mmol) of β-propiolactone (Tokyo Chemical Industry Co., Ltd.) and 0.0019 g (0.016 mmol) of N,N,N-trimethylglycine carboxylate (Tokyo Chemical Industry Co., Ltd.) were dissolved in 11.8 g of tetrahydrofuran (hereinafter referred to as THF) and stirred at 50 °C for 8 hours. Thereafter, the mixture was cooled to room temperature and allowed to stand for one week, and the precipitated solid was filtered and dried to obtain 0.52 g of raw material (C) containing p-3P polymer. The weight-average molecular weight (Mw) of raw material (C) was measured by gel permeation chromatography (GPC) and found to be 20,700. From this result, raw material (C) is determined to be a 287 mer.

### Preparation Example 4: Synthesis of Raw Material (D) Containing 3-HPA Polymer

5.00 g (63.39 mmol) of β-propiolactone and 0.0081 g (0.0694 mmol) of N,N,N-trimethylglycine carboxylate were dissolved in 50 g of THF and allowed to stand at room temperature for two months. Thereafter, the precipitated solid was then filtered and dried to obtain 1.76 g of raw material (D) containing p-3HP polymer. The Mw of raw material (D) was measured in the same manner as in Preparation Example 3, and was found to be 74,600. From this result, raw material (D) is deemed to be a 1,035 mer.

### Preparation Example 5: Synthesis of Raw Material (E) Containing 3-HPA Polymer

20.00 g (277.55 mmol) of β-propiolactone and 0.0406 g (0.3469 mmol) of N,N,N-trimethylglycine carboxylate were dissolved in 300 g of THF and stirred at 50 °C for 50 hours. The mixture was then cooled to room temperature and allowed to stand for two months. The precipitated solid was filtered and dried to obtain 0.52 g of raw material (E) containing p-3HP polymer. The Mw of raw material (E) was measured in the same manner as in Preparation Example 3, and was found to be 153,700. From this result, raw material (E) is deemed to be a 2,133 mer.

### Preparation Example 6: Synthesis of Raw Material (F) Containing 3-HPA Polymer

25.00 g (364.94 mmol) of β-propiolactone and 0.0406 g (0.3469 mmol) of N,N,N-trimethylglycine carboxylate were dissolved in 200 g of THF and left to stand at room temperature for two months. The precipitated solid was filtered and dried to obtain 9.55 g of raw material (F) containing p-3HP polymer. The obtained raw material (F) was not soluble in THF or dimethylformamide, and it was not possible to measure the Mw by GPC measurement using these as the mobile phase. Therefore, the filtrate obtained when filtering out the raw material (F) was measured by GPC under the same conditions as described above, and the Mw was 643,000. Therefore, it is considered that the Mw of the obtained solid p-3HP is at least 643,000 or more. Furthermore, from this result, it is determined that the raw material (F) is at least an 8,922 mer.

### Reference Example 1: Measurement of Pyrolysis Temperature of Each Raw Material

The pyrolysis temperature at which the weight loss is 5% was measured for each of the above raw materials using a simultaneous thermogravimetry and differential thermal analysis (TG-DTA) device. The pyrolysis temperature at which the weight loss is 5% is considered to be the temperature at which 5% of the 3-HPA contained in the respective raw materials is acrylic acid. The measurement conditions and results are as follows.

### <Measurement Conditions>

### Measurement Device:

Heating conditions: 50 °C to 100 °C; 10 °C/min, 100 °C; 5 min, 100 °C to 400 °C; 10 °C/min

### <Measurement Results>

Raw material (A): 165 °C
Raw material (B): 175 °C
Raw material (C): 232 °C
Raw material (D): 237 °C
Raw material (E): 243 °C
Raw material (F): 266 °C

From the above measurement results, it is understood that in the case where a basic catalyst and a carboxylic acid anhydride are not contained, high temperature and long time conditions are required to produce acrylic acid from the raw materials (A) to (F) containing a 3-HPA polymer.

### Example 1

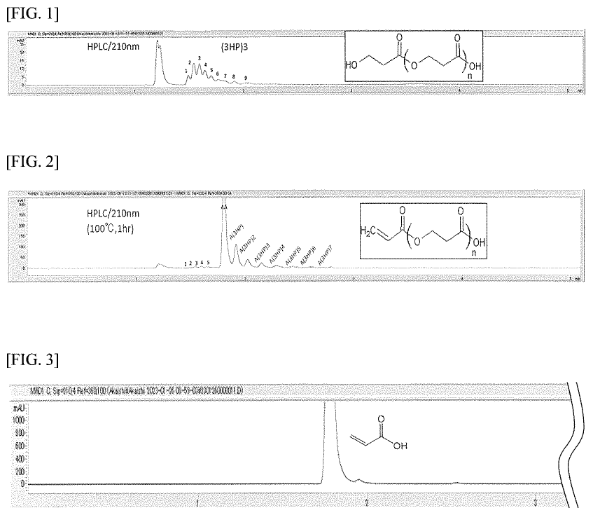

240 mg of raw material (A) containing 3-HPA and polymer thereof obtained in Preparation Example 1, 5.7 mg of potassium acrylate (manufactured by Nippon Shokubai Co., Ltd.), 50.3 mg of acrylic anhydride (manufactured by Osaka Organic Chemical Industry Co., Ltd.), and 0.3 mg of polymerization inhibitor (commercially available 4-hydroxy-TEMPO) were placed in a 10 mL pressure-resistant container and heated at 100 °C for 1 hour while stirring to obtain 296 mg of reaction product (1). The obtained reaction product (1) was analyzed by HPLC, and the amounts of (a) acrylic acid, (b) A⁻ 3-HPA polymer, (c) 3-HPA polymer, and (d) 3-HPA, which are represented by the following chemical formulas, contained in the reaction product (1) were quantified. However, for (a), acrylic acid (manufactured by Tokyo Kaseihin Kogyo Co., Ltd.) was used as a standard. The quantitative results are shown in Table 1, and the HPLC chart is shown in FIG. 2.

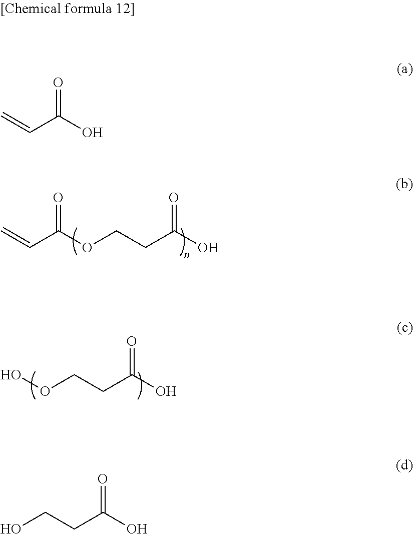

In the HPLC analysis of the product of Example 1, the reaction product (1) was completely dissolved in the mobile phase, and no insoluble matter was found. Since the obtained acrylic acid includes those derived from the raw material (A) as well as those derived from potassium acrylate and acrylic anhydride, the yield in this example was determined as follows:

Yield (%)=(weight of acrylic acid contained in the reaction product)/(weight of raw material (A)+weight of potassium acrylate+weight of acrylic anhydride)

As can be seen from the comparison between FIG. 1 and FIG. 2, when comparing the distribution of the degree of polymerization in A-3-HPA polymer contained in the reaction product (1) and the distribution of the degree of polymerization of 3-HPA polymer in the raw material (A), in the reaction product (1), the content of a lower degree of polymerization was larger than that of the raw material (A). This indicates that the reaction has occurred as shown in Reaction Scheme (2) in which the ester-bond of 3-HPA polymer is cleaved to produce acrylate.

### Example 2

The reaction product (2) according to Example 2 was produced in the same manner as in Example 1, except that the reaction product (2) was further heated at 150 °C for 1 hour after heating at 100 °C for 1 hour. The results are shown in Table 1.

### Example 3

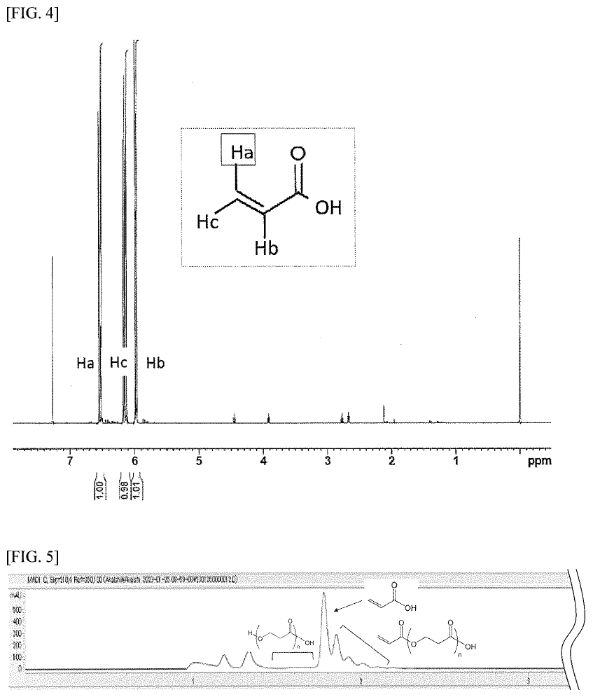

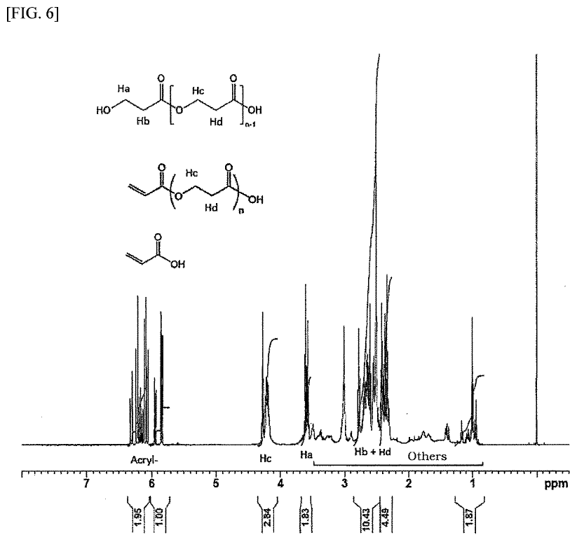

1000 mg of the raw material (A) obtained in Preparation Example 1, 24 mg of potassium acrylate (pKa of acrylic acid 4.26), 200 mg of acrylic anhydride, and 1 mg of a polymerization inhibitor (4-hydroxy-TEMPO) were placed in a Kugelrohr sample ball, and while rotating under reduced pressure of 25 KPa to 18 KPa and a jacket temperature of 125 °C heated to 125 °C. A transparent distillate was obtained by heating while raising the temperature from then to 194 °C in 2 hour. When the distillate was analyzed by HPLC and ¹H-NMR, it was found to contain 620 mg of acrylic acid (GC content 96.6%). The HPLC and ¹H-NMR charts of the distillate are shown in FIGS. 3 and 4, respectively. In the HPLC charts, no peaks other than those shown in FIG. 3 were detected. Furthermore, 350 mg of reaction product (3) was produced in the sample ball, and reaction product (3) was analyzed by HPLC and ¹H-NMR. The analysis results are shown in Table 1, and the HPLC and ¹H-NMR charts of reaction product (3) are shown in FIGS. 5 and 6, respectively. No peaks other than those shown in FIG. 5 were detected in the HPLC chart. The yield was calculated as follows: Yield (%)=(weight of acrylic acid contained in the reaction product+weight of acrylic acid in the distillate)/(weight of raw material (A)+weight of potassium acrylate+weight of acrylic anhydride)

### Example 4

A distillate and a reaction product (4) were obtained and analyzed in the same manner as in Example 3, except that the pressure was changed from 20 KPa to 18 KPa and other conditions and compositions were changed to those shown in Table 1. The analysis results are shown in Table 1.

### Comparative Examples 1 to 3

Preparation of acrylic acid and analysis of comparative reaction products (1) to (3) were carried out in the same manner as in Example 1 except that the conditions were changed as shown in Table 1. The results are shown in Table 1.

TABLE 1

| | | Example | | | | Comparative Example | | |
|---|---|---|---|---|---|---|---|---|
| | | 1 | 2 | 3 | 4 | 1 | 2 | 3 |
| Composition | Raw Material (A) | 240 | 240 | 1000 | — | 240 | 240 | 246 |
| | Raw Material (B) | — | — | — | 1000 | — | — | — |
| | Potassium Acrylate | 5.7 | 5.7 | 24 | 19 | 4.8 | 5.7 | — |
| | Acrylic Acid Anhydride | 50.3 | 50.3 | 200 | 131 | — | — | — |
| Reaction | Temperature [°C] | 100 | 100 to 150 | 125 to 194 | 125 to 200 | 220 | 100 | 100 |
| conditions | Time [hr] | 1 | 1 to 1 | 2 (gradual | 2 (gradual | 1 | 1 | 1 |
| | | | | heating) | heating) | | | |
| Total amount of Reaction Product [mg] | | 296 | 296 | 350 | 275 | 245 | 246 | 246 |
| Composition [mg] | Acrylic Acid | 40 | 38 | 31 | 35 | 2 | 3 | 0 |
| | A-3-HPA Polymer | 62 | 101 | 43 | 32 | 3 | 0.5 | 0.5 |
| | 3-HPA Polymer | 183 | 23 | 46 | 24 | 3 | 180 | 245 |
| | 3-HPA | 10 | 7 | 0 | 0 | 1 | 21 | 0 |
| | Others | 1 | 127 | 230 | 184 | 236 | 42 | 0.5 |
| Distillate [mg] | Acrylic Acid | — | — | 620 | 809 | — | — | — |
| Acrylic Acid | Generation [mg] | 40 | 38 | 651 | 844 | 2 | 3 | 0 |
| | Yield [%] | 14 | 13 | 53 | 73 | 1 | 1 | 0 |

| | | | | | | | |
|---|---|---|---|---|---|---|---|
| Total Loaded Weight [mg] | 296 | 296 | 1,224 | 1,150 | 245 | 246 | 246 |

From Table 1, it was confirmed that the reaction proceeded even at a relatively low temperature and acrylic acid was produced according to the production method of the present invention described in Examples 1 to 4. In each Examples, the heating was stopped before the reaction was completely completed in order to check the progress of the reaction, and 3-HPA polymer remained in the reaction product. Therefore, the reaction yield can be further improved by, for example, extending the reaction time. In contrast, in the cases of Comparative Examples 1 to 3, almost no acrylic acid, which is the target product, was obtained.

In the following Examples and Comparative Examples, the weight loss rate when the reaction was carried out under the following conditions was measured by a TG-DTA apparatus and was considered to correspond to the yield.

### Example 5

100 parts by mass of raw material (B) containing 3-HPA and polymer thereof obtained in Preparation Example 2, 2 parts by mass of potassium acrylate (manufactured by Nippon Shokubai Co., Ltd.), and 22 parts by mass of acrylic anhydride (manufactured by Osaka Organic Chemical Industry Ltd.) were placed in an aluminum container in an amount of 10 mg, heated from 50 °C to 100 °C over the course of 5 minutes, then maintained at 100 °C for 5 minutes, heated to 150 °C over the course of 5 minutes, and heated at 150 °C for 55 minutes (heating condition 1). The heating time from 50 to 150 °C was 70 minutes in total. Based on the weight loss rate, the yield was corresponded to 66.1%.

### Example 6-1

The reaction was carried out in the same manner as in Example 5, except that the raw material (B) was replaced with the raw material (C) (Mw: 20,700) containing 3-HPA and polymer thereof obtained in Preparation Example 3, and acrylic anhydride was not used. From the weight loss rate, the yield was equivalent to 22.4%.

### Example 6-2

The reaction was carried out in the same manner as in Example 6-1 except that 5 parts by mass of acrylic anhydride was further used. From the weight loss rate, the yield corresponds to 56.0%.

### Example 6-3

The reaction was carried out in the same manner as in Example 6-2 except that the amount of acrylic anhydride was changed to 10 parts by mass. From the weight loss rate, the yield corresponds to 85.5%.

### Example 6-4

The reaction was carried out in the same manner as in Example 6-2 except that the amount of acrylic anhydride was changed to 25 parts by mass. From the weight loss rate, the yield corresponds to 95.5%.

### Example 6-5

The reaction was carried out in the same manner as in Example 6-1 except that sodium 3-hydroxypropionate (manufactured by Cosmo Bio Co., Ltd.) was used instead of potassium acrylate. From the weight loss rate, the yield corresponds to 10.2%.

### Example 6-6

The reaction was carried out in the same manner as in Example 6-1, except that and 2 parts by mass of sodium 3-hydroxypropionate (manufactured by Cosmo Bio Co., Ltd.) were further used. From the weight loss rate, the yield corresponds to 35.0%.

### Example 6-7

The reaction was carried out in the same manner as in Example 6-6, except that 9 parts by mass of acrylic anhydride was further used. From the weight loss rate, the yield corresponds to 54.0%.

### Example 6-8

The reaction was carried out in the same manner as in Example 6-1 except that the amount of potassium acrylate was changed to 6 parts by mass. From the weight loss rate, the yield corresponds to 65.7%.

### Example 6-9

The reaction was carried out in the same manner as in Example 6-1 except that 5 parts by mass of potassium carbonate (K carbonate) was used instead of potassium acrylate. From the weight loss rate, the yield corresponds to 65.0%.

### Comparative Example 5

### Comparative Example 5

The reaction was carried out in the same manner as in Example 5, except that only 100 parts by mass of raw material (B) containing 3-HPA and polymer thereof obtained in Preparation Example 2 was used. From the weight loss rate, the yield corresponds to 0.6%.

### Comparative Example 6

The reaction was carried out in the same manner as in Comparative Example 5 except that only 3-HPA obtained in Preparation Example 3 and the raw material (C) containing the polymer were used instead of the raw material (B).

From the weight loss, the yield corresponds to 0.6%.

### Example 7-1

The reaction was carried out in the same manner as in Example 5, except that raw material (D) (Mw: 74,600) containing 3-HPA and polymer thereof obtained in Preparation Example 4 was used instead of raw material (B), the amount of potassium acrylate was 5 parts by mass, and acrylic anhydride was not used. From the weight loss rate, the yield corresponds to 41.0%.

### Example 7-2a

The reaction was carried out in the same manner as in Example 7-1 except that 10 parts by mass of acrylic anhydride was further used. From the weight loss rate, the yield corresponds to 87.8%.

### Example 7-2b

The reaction product 7-2b was obtained in the same manner as in Example 7-2a, except that instead of heating condition 1, the mixture in the pressure-resistant container was heated with stirring, heated from 50 °C to 100 °C over the course of 5 minutes, then maintained at 100 °C for 5 minutes, then heated to 150 °C over the course of 5 minutes, and heated at 150 °C for 85 minutes (heating condition 2). The heating time from 50 to 150 °C under heating condition 2 was 100 minutes in total. From the weight loss rate, the yield corresponds to 97.2%.

### Example 7-2c

The reaction product 7-2c was obtained in the same manner as in Example 7-2a, except that the mixture contained in the pressure-resistant vessel was heated with stirring, heated from 50 °C to 100 °C over the course of 5 minutes, then maintained at 100 °C for 5 minutes, then heated to 150 °C over the course of 5 minutes, and then heated at 150 °C for 135 minutes (heating condition 3). The heating time at 50-150 °C under heating condition 3 was a total of 150 minutes. From the weight loss rate, the yield corresponds to 98.3%.

### Example 7-3a to 7-3c

Analyses were carried out in the same manner as in 7-2a to 7-2c except that 5 parts by mass of potassium carbonate was used instead of potassium acrylate and no acrylic anhydride was used. From the weight loss ratio, the yield of 7-3a corresponds to 55.7%, the yield of 7-3b to 71.4%, and the yield of 7-3c to 80.9%.

### Example 8-1

The reaction was carried out in the same manner as in Example 5 except that instead of the raw material (B), raw material (E) (Mw: 153,700) containing 3-HPA and polymer thereof obtained in Preparation Example 5 was used, the amount of potassium acrylate was 5 parts by mass, and acrylic anhydride was not used. From the weight loss ratio, the yield corresponds to 48.3%.

### Example 8-2a

The reaction was carried out in the same manner as in Example 8-1 except that 10 parts by mass of acrylic anhydride was further used. From the weight loss ratio, the yield corresponds to 91.0%.

### Example 8-2b

The reaction was carried out in the same manner as in Example 8-2a except that the heating was carried out under the heating condition 2 instead of the heating condition 1. From the weight loss ratio, the yield corresponds to 97.2%.

### Example 8-2c

The reaction was carried out in the same manner as in Example 8-2a except that the heating was carried out under the heating condition 3 instead of the heating condition 1. From the weight loss ratio, the yield corresponds to 97.2%.

### Example 8-3a~8-3c

The reaction was carried out in the same manner as in Example 8-2a to 8-2c except that 5 parts by mass of potassium carbonate was used instead of potassium acrylate and no acrylic anhydride was used. From the weight loss ratio, the yield of 8-3a corresponds to 59.9%, the yield of 8-3b corresponds to 89.3%, and the yield of 8-3c corresponds to 97.7%.

### Example 9-1

The reaction was carried out in the same manner as in Example 6 except that, instead of the raw material (B), using the raw material (F) containing 3-HPA and polymer thereof obtained in Preparation Example 5 (Mw: at least 643,000), the quantity of potassium acrylate and 5 parts by weight, and acrylic anhydride was not used. From the weight loss ratio, the yield corresponds to 34.7%.

### Example 9-2a to 9-2c

The reaction was carried out in the same manner as in Example 9-1, except that 11 parts by mass of acrylic anhydride was further used. From the weight loss ratio, the yield of acrylate in 9-2a corresponds to 50.2%. The reaction was carried out in the same manner as described above except that the heating condition 1 was changed to the heating condition 2. From the weight loss ratio, the yield of acrylate in 9-2b corresponds to 77.3%. Analysis was carried out in the same manner as described above except that the heating condition 1 was changed to the heating condition 3. From the weight loss ratio, the yield of acrylate in 9-2c corresponds to 98.2%.

### Example 9-3a to 9-3c

The reaction was carried out in the same manner as in Example 9-2a to 9-2c except that the amount of acrylic anhydride was changed to 10 parts by mass and zinc acrylate (II) was used instead of potassium acrylate. From the weight loss ratio, the yield of 9-3a corresponds to 46.0%, the yield of 9-3b corresponds to 77.4%, and the yield of 9-3c corresponds to 95.1%.

### Example 9-4a to 9-4c

The reaction was carried out under heating conditions 1 to conditions 3 in the same manner as in Example 9-1 except that potassium carbonate was used instead of potassium acrylate. From the weight loss ratio, the yield of 9-4a corresponds to 67.2%, the yield of 9-4b corresponds to 92.4%, and the yield of 9-4c corresponds to 96.6%.

### Example 9-5a to 9-5c

The reaction was carried out under heating conditions 1 to conditions 3 in the same manner as in Example 9-1, except that sodium carbonate was used instead of potassium acrylate. From the weight loss ratio, the yield of 9-5a corresponds to 41.0%, the yield of 9-5b corresponds to 68.3%, and the yield of 9-5c corresponds to 93.5%.

### Example 9-6a to 9-6c

The reaction was carried out in the same manner as in Example 9-5a to 9-5c except that 20 parts by weight water was added. From the weight loss ratio, the yield of 9-6a corresponds to 33.8%, the yield of 9-6b corresponds to 58.8%, and the yield of 9-6c corresponds to 76.3%.

### Example 10

The raw material (F) 0.500 g and the potassium carbonate 0.025 g were mixed in a vessel and placed in a vacuum distiller. When the mixture was heated for about 140 minutes under the conditions of bath temperature: 100 to 155 °C, internal pressure: 20.6 to 16.0 kPa, gas considered to be carbon dioxide was generated in the early stages. Thereafter, a fraction of 0.470 g was obtained. GC analysis of the fraction found that it contained 97.0% acrylic acid, with a yield of 94%.

The conditions and results of Examples 5 to 9 and Comparative Examples 5 to 6 above are summarized in Tables 2 and 3.

TABLE 2

| | | | | | | | | | | | | | Comparative | |
|---|---|---|---|---|---|---|---|---|---|---|---|---|---|---|
| | Molecular | Degree of | Example | | | | | | | | | | Example | |
| | Weight | polymerization | 5 | 6-1 | 6-2 | 6-3 | 6-4 | 6-5 | 6-6 | 6-7 | 6-8 | 6-9 | 5 | 6 |
| Raw | 234 | 3 | | | | | | | | | | | | |
| Material(A) | | | | | | | | | | | | | | |
| Raw | 522 | 7 | 100 | | | | | | | | | | 100 | |
| Material(B) | | | | | | | | | | | | | | |
| Raw | 20,700 | 287 | | 100 | 100 | 100 | 100 | 100 | 100 | 100 | 100 | 100 | | 100 |
| Material(C) | | | | | | | | | | | | | | |
| Raw | 74,600 | 1,035 | | | | | | | | | | | | |
| Material(D) | | | | | | | | | | | | | | |
| Raw | 153,700 | 2,133 | | | | | | | | | | | | |
| Material(E) | | | | | | | | | | | | | | |
| Raw | 643,000 | 8,922 | | | | | | | | | | | | |
| Material(F) | | | | | | | | | | | | | | |

| | | | | | | | | | | | | | |
|---|---|---|---|---|---|---|---|---|---|---|---|---|---|
| Basic | Potassium acrylate | 2 | 2 | 2 | 2 | 2 | | 2 | 2 | 6 | | | |
| Catalyst | Sodium 3- | | | | | | 2 | 2 | 2 | | | | |
| | hydroxypropionate | | | | | | | | | | | | |
| | K carbonate | | | | | | | | | | 5 | | |
| Carboxylic | Acrylic Anhydride | 22 | | 5 | 10 | 25 | | | 9 | | | | |
| Anhydride | | | | | | | | | | | | | |
| Yiled(%) | Heating condition 1 | 66.1 | 22.4 | 56.0 | 85.5 | 95.5 | 10.2 | 35.0 | 54.0 | 65.7 | 65.0 | 0.6 | 0.6 |

TABLE 3

| | Molecular | Degree of | Example | | | | | | | | | | | |
|---|---|---|---|---|---|---|---|---|---|---|---|---|---|---|
| | Weight | polymerization | 7-1 | 7-2 | 7-3 | 8-1 | 8-2 | 8-3 | 9-1 | 9-2 | 9-3 | 9-4 | 9-5 | 9-6 |
| Raw | 234 | 3 | | | | | | | | | | | | |
| Material(A) | | | | | | | | | | | | | | |
| Raw | 522 | 7 | | | | | | | | | | | | |
| Material(B) | | | | | | | | | | | | | | |
| Raw | 20,700 | 287 | | | | | | | | | | | | |
| Material(C) | | | | | | | | | | | | | | |
| Raw | 74,600 | 1,035 | 100 | 100 | 100 | | | | | | | | | |
| Material(D) | | | | | | | | | | | | | | |
| Raw | 153,700 | 2,133 | | | | 100 | 100 | 100 | | | | | | |
| Material(E) | | | | | | | | | | | | | | |
| Raw | 643,000 | 8,922 | | | | | | | 100 | 100 | 100 | 100 | 100 | 100 |
| Material(F) | | | | | | | | | | | | | | |
| Basic | Potassium acrylate | | 5 | 5 | | 5 | 5 | | 5 | 5 | | | | |
| Catalyst | Zinc acrylate (II) | | | | | | | | | | 5 | | | |
| | K carbonate | | | | 5 | | | 5 | | | | 5 | | |
| | Sodium carbonate | | | | | | | | | | | | 5 | 5 |
| Carboxylic | Acrylic Anhydride | | | 10 | | | 10 | | | 11 | 10 | | | |
| Anhydride | | | | | | | | | | | | | | |

| Water | | | | | | | | | | | | | 20 |
|---|---|---|---|---|---|---|---|---|---|---|---|---|---|
| Yiled(%) | Heating condition 1 | 41.0 | 87.8 | 55.7 | 48.3 | 91.0 | 59.9 | 34.7 | 50.2 | 46.0 | 67.2 | 41.0 | 33.8 |
| | Heating condition 2 | — | 97.2 | 71.4 | — | 97.2 | 89.3 | — | 77.3 | 77.4 | 92.4 | 68.3 | 58.8 |
| | Heating condition 3 | — | 98.3 | 80.9 | — | 97.2 | 97.7 | — | 98.2 | 95.1 | 96.6 | 93.5 | 76.3 |

According to the production methods of the present invention described in Examples 5 to 9, it was confirmed from Tables 2 and 3 that the reaction proceeded and acrylic acid was produced even at a relatively low temperature of about 150 °C.

For example, in the case of Example 5, 6-2 to 6-4, 6-7, 7-2, 8-2, and 9-2 to 9-3 in which the mixture containing the 3-hydroxycarboxylic acid and/or polymer thereof and the basic catalyst further contained the carboxylic acid anhydride, acrylic acid could be obtained regardless of the molecular weight of the raw material.

In addition, in the case of Examples 6 to 9 in which the weight-average molecular weight of the polymer of the 3-hydroxycarboxylic acid contained in the raw material was 10,000 or more, acrylic acid could be obtained even when no carboxylic acid anhydride was further contained. In contrast, in Comparative Examples 5 and 6, almost no acrylic acid as the target product was obtained. Furthermore, when Example 9-5 and Example 9-6 are compared, the yield of the target product acrylic acid was higher in Example 9-5, in which the mixture contained less water, than in Example 9-6, in which the mixture contained water. In addition, when comparing Example 9-5c and Example 10, which have the same types and ratios of raw materials and basic catalysts, it was confirmed that the weight loss rate in Example 9-5 and the yield of acrylic acid measured by GC analysis in Example 10 were almost the same. Therefore, it can be said that the weight loss rate in each of the above examples corresponds to the yield.

1. A method for producing a (meth)acrylic acid compound using a hydroxycarboxylic acid and/or a polymer thereof as a raw material, comprising at least a reaction step of heating a mixture containing a hydroxycarboxylic acid and/or a polymer thereof and a basic catalyst to react with each other to produce a (meth)acrylic acid compound, wherein (1) the mixture further contains a carboxylic acid anhydride, and/or (2) the weight-average molecular weight of the polymer of the hydroxycarboxylic acid contained in the raw material is 10,000 or more.

2. The method according to claim 1, a method for producing a (meth)acrylic acid compound using a 3-hydroxycarboxylic acid and/or a polymer thereof as a raw material, comprising at least a reaction step of heating a mixture containing a 3-hydroxycarboxylic acid and/or a polymer thereof, a basic catalyst, and a carboxylic acid anhydride to react with each other to produce a (meth)acrylic acid compound.

3. The method according to claim 1, wherein the hydroxycarboxylic acid is at least one selected from the group consisting of 2-hydroxycarboxylic acid and 3-hydroxycarboxylic acid.

4. The method according to claim 2, wherein the carboxylic acid anhydride is (meth)acrylic acid anhydride.

5. The method according to claim 4, wherein the (meth)acrylic anhydride is represented by the formula (I): 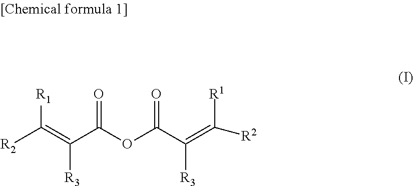
wherein
R¹ to R³ represent, each independently, a hydrogen atom or an alkyl group having 1 to 3 carbon atoms.

6. The method according to claim 1, wherein the basic catalyst is selected from the group consisting of an alkali metal carboxylate, a quaternary ammonium carboxylate, and an ammonium betaine.

7. The method according to claim 1, wherein the basic catalyst is at least one alkali metal carboxylate selected from the group consisting of an alkali metal salt of an α,β-ethylenically unsaturated monocarboxylic acid and an alkali metal salt of an aliphatic monocarboxylic acid.

8. The method according to claim 1, wherein the basic catalyst is at least one alkali metal carboxylate selected from the group consisting of alkali metal (meth)acrylate and alkali metal acetate.

9. The method according to claim 1, wherein the amount of the basic catalyst is 0.1 to 20 parts by mass per 100 parts by mass of the mixture including the hydroxycarboxylic acid and its polymer.

10. The method according to claim 2, wherein the amount of the carboxylic acid anhydride is 1 to 50 parts by mass per 100 parts by mass of the total of the hydroxycarboxylic acid and the polymer thereof in the mixture.

11. The method according to claim 1, wherein the heating temperature in the reaction step is 80 to 220 °C.

12. The process according to claim 2, wherein the hydroxycarboxylic acid is 3-hydroxypropionic acid, and the carboxylic acid anhydride is a (meth)acrylic acid anhydride represented by the formula (I) in which R¹ to R³ is a hydrogen atom, or R¹ and R² are a hydrogen atom, and R³ represents a methyl group.

13. The method according to claim 1, wherein the mixture further comprises a polymerization inhibitor.

14. The method according to claim 1, wherein the content of the solvent in the mixture is 0 to 50% by mass based on the total amount of the mixture.

15. The method according to claim 1, wherein the weight-average molecular weight of the polymer of the hydroxycarboxylic acid contained in the raw material is 10,000 or more, and the content of water in the mixture is 0 to 15% by mass based on the total amount of the mixture.

16. The method according to claim 1, further comprising a step of distilling off the (meth)acrylic acid compound produced in the reaction step.
